(19) United States
(12) Patent Application Publication
(10) Pub. No.: US 2025/0163034 A1
(43) Pub. Date: May 22, 2025

(54) ORGANIC COMPOUND AND ORGANIC LIGHT-EMITTING ELEMENT

(71) Applicant: CANON KABUSHIKI KAISHA, Tokyo (JP)

(72) Inventors: HIRONOBU IWAWAKI, Kanagawa (JP); TAKAYOSHI KIKUCHI, Kanagawa (JP); YOSUKE NISHIDE, Kanagawa (JP); HIROKI OHRUI, Tokyo (JP)

(21) Appl. No.: 18/946,397

(22) Filed: Nov. 13, 2024

(30) Foreign Application Priority Data
Nov. 21, 2023 (JP) 2023-197266

(51) Int. Cl. C07D 409/14; C07B 59/00; C07D 405/10; C07D 405/14; C09K 11/06; H10K 50/16; H10K 85/20; H10K 85/30; H10K 85/60

(52) U.S. Cl. CPC C07D 409/14; C07B 59/004; C07D 405/10; C07D 405/14; C09K 11/06; H10K 50/16; H10K 85/20; H10K 85/324; H10K 85/622; H10K 85/653; H10K 85/654; H10K 85/6572; H10K 85/6576; C07B 2200/05

(57) ABSTRACT

As an organic light-emitting element with high durability, an organic light-emitting element in which an organic compound with an indolocarbazole ring and a xanthone ring bonded through a benzene ring, a dibenzothiophene ring, or a xanthone ring is used as a host of a light-emitting layer.

### BACKGROUND OF THE INVENTION

### Field of the Invention

The present disclosure relates to a novel organic compound and an organic light-emitting element using the organic compound.

### Description of the Related Art

An organic light-emitting element (hereinafter referred to as an “organic electroluminescent element” or an “organic EL element”) is an electronic element that includes a pair of electrodes and an organic compound layer between the electrodes. Electrons and holes are injected from the pair of electrodes to generate an exciton of a light-emitting organic compound in the organic compound layer. When the exciton returns to its ground state, the organic light-emitting element emits light.

With recent significant advances in organic light-emitting elements, it is possible to realize low drive voltage, various emission wavelengths, high-speed responsivity, and thin and lightweight light-emitting devices.

Compounds suitable for organic light-emitting elements have been actively developed. This is because development of a compound with good element life characteristics is important to provide a high-performance organic light-emitting element. An indolocarbazole derivative 1-a in Chinese Patent Application Publication No. 113861173 (hereinafter PTL1) and 1-b in Chinese Patent Application Publication No. 114249734 (hereinafter PTL2) are described as compounds that have been developed.

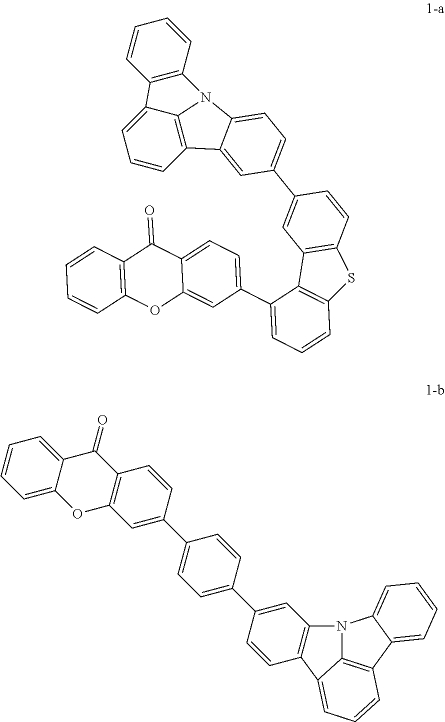 [SMILES: O=c1c2ccccc2oc2cc(-c3cccc4sc5ccc(-c6ccc7c(c6)c6cccc8c9ccccc9n7c86)cc5c34)ccc12 ; O=c1c2ccccc2oc2cc(-c3ccc(-c4ccc5c6cccc7c8ccccc8n(c5c4)c76)cc3)ccc12]

Although PTL 1 and PTL 2 disclose an organic compound for use in an organic light-emitting element with good element life characteristics, further improvement in durability is desired in the organic light-emitting element.

### SUMMARY OF THE INVENTION

In view of these disadvantages, the present disclosure provides an organic light-emitting element with high durability.

An organic compound according to the present disclosure is represented by formula [1]:

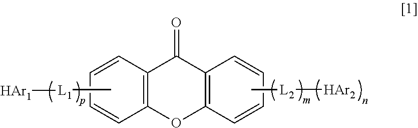

wherein p, m, and n are each independently 0 or 1, and p+m+n≥1,
L₁ is a divalent substituent derived from a substituted or unsubstituted biphenyl, terphenyl, dibenzothiophene, or combination of benzenes, dibenzothiophenes, and xanthones, wherein in a case of a number of the benzenes is q, a number of dibenzothiophenes is r, and a number of xanthones is s, each of the q, the r and the s independently is an integer of 0 or 1 or more, and q+r+s≥2,
L₂ is a monovalent or divalent substituent derived from a substituted or unsubstituted benzene, biphenyl, terphenyl, dibenzothiophene, or combination of benzenes, dibenzothiophenes, and xanthones, wherein in a case of a number of the benzenes is q, a number of dibenzothiophenes is r, and a number of xanthones is s, each of the q, the r and the s independently is an integer of 0 or 1 or more, and q+r+s≥2, and
HAr₁ and HAr₂ are each independently represented by any one of formulae [2-1] to [2-3],

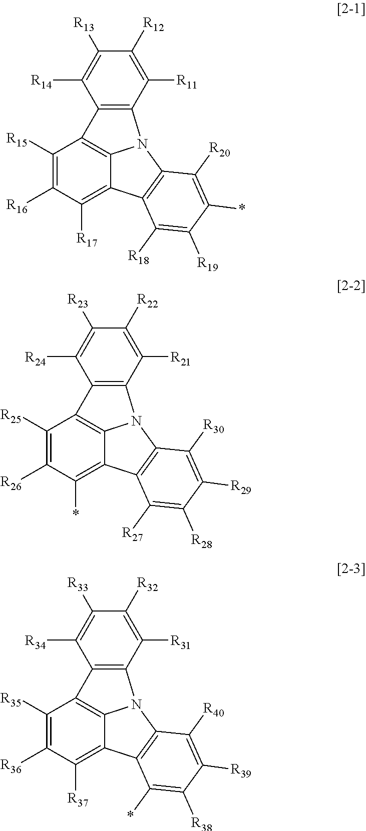

wherein R₁₀ to R₄₀ are selected from a hydrogen atom, a deuterium atom, a halogen atom, a substituted or unsubstituted alkyl group, a substituted or unsubstituted alkoxy group, and a substituted or unsubstituted silyl group.

An organic light-emitting element according to the present disclosure includes a first electrode, a second electrode, and an organic compound layer between the first electrode and the second electrode,
wherein the organic compound layer contains the organic compound according to the present disclosure.

Further features of the present disclosure will become apparent from the following description of exemplary embodiments with reference to the attached drawings.

### BRIEF DESCRIPTION OF THE DRAWINGS

FIG. 1A is a schematic cross-sectional view of an example of a pixel of a display apparatus according to an embodiment of the present disclosure.

FIG. 1B is a schematic cross-sectional view of an example of a display apparatus including an organic light-emitting element according to an embodiment of the present disclosure.

FIG. 2 is a schematic view of an example of a display apparatus according to an embodiment of the present disclosure.

FIG. 3A is a schematic view of an example of an imaging apparatus according to an embodiment of the present disclosure.

FIG. 3B is a schematic view of an example of electronic equipment according to an embodiment of the present disclosure.

FIG. 4A is a schematic view of an example of a display apparatus according to an embodiment of the present disclosure.

FIG. 4B is a schematic view of an example of a foldable display apparatus.

FIG. 5A is a schematic view of an example of a lighting apparatus according to an embodiment of the present disclosure.

FIG. 5B is a schematic view of an example of an automobile with a vehicle lamp according to an embodiment of the present disclosure.

FIG. 6A is a schematic view of an example of a wearable device according to an embodiment of the present disclosure.

FIG. 6B is a schematic view of an example of a wearable device according to an embodiment of the present disclosure including an imaging apparatus.

FIG. 7A is a schematic view of an example of an image-forming apparatus according to an embodiment of the present disclosure.

FIGS. 7B and 7C are schematic views of an exposure light source for an image-forming apparatus according to an embodiment of the present disclosure.

### DESCRIPTION OF THE EMBODIMENTS

Embodiments of the present disclosure are described below. The present disclosure is not limited to the following description, and it is easily understood by those skilled in the art that modes and details thereof can be modified in various ways without departing from the gist and scope of the present disclosure. Thus, the present disclosure is not construed as being limited by the following description.

### [Organic Compound]

First, an organic compound according to the present embodiment is described below. An organic compound according to the present disclosure is a compound represented by formula [1]:

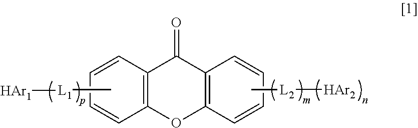

wherein p, m, and n are each independently 0 or 1, and p+m+n≥1,
L₁ is a divalent substituent derived from a substituted or unsubstituted biphenyl, terphenyl, dibenzothiophene, or combination of benzenes, dibenzothiophenes, and xanthones, wherein in a case of a number of the benzenes is q, a number of dibenzothiophenes is r, and a number of xanthones is s, each of the q, the r and the s independently is an integer of 0 or 1 or more, and q+r+s≥2,
L₂ is a monovalent or divalent substituent derived from a substituted or unsubstituted benzene, biphenyl, terphenyl, dibenzothiophene, or combination of benzenes, dibenzothiophenes, and xanthones, wherein in a case of a number of the benzenes is q, a number of dibenzothiophenes is r, and a number of xanthones is s, each of the q, the r and the s independently is an integer of 0 or 1 or more, and q+r+s≥2, and
HAr₁ and HAr₂ are each independently represented by any one of formulae [2-1] to [2-3],

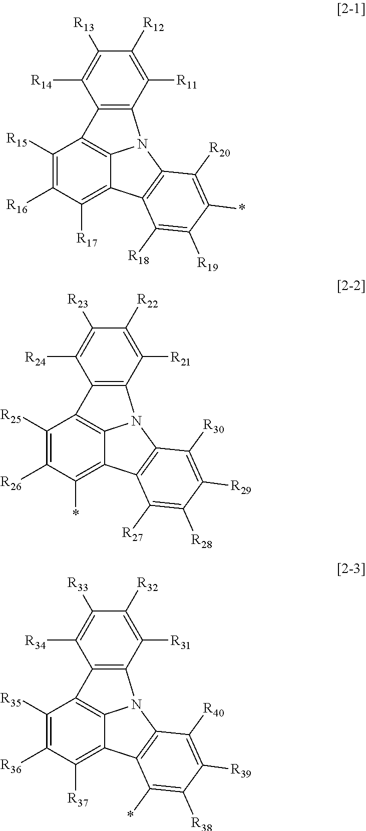

wherein R₁₀ to R₄₀ are selected from a hydrogen atom, a deuterium atom, a halogen atom, a substituted or unsubstituted alkyl group, a substituted or unsubstituted alkoxy group, and a substituted or unsubstituted silyl group.

The halogen atom, the alkyl group, the alkoxy group, and the silyl group mentioned as R₁₀ to R₄₀ are more specifically described below.

The halogen atom is, for example, but not limited to, fluorine, chlorine, bromine, iodine, astatine, tennessine, or the like.

The alkyl group may be an alkyl group with 1 or more and 20 or less carbon atoms. The alkyl group is, for example, but not limited to, a methyl group, an ethyl group, a n-propyl group, an isopropyl group, a n-butyl group, a tert-butyl group, a sec-butyl group, an octyl group, a cyclohexyl group, a tert-pentyl group, a 3-methylpentan-3-yl group, a 1-adamantyl group, a 2-adamantyl group, or the like.

The alkoxy group may be an alkoxy group with 1 or more and 10 or less carbon atoms. The alkoxy group is, for example, but not limited to, a methoxy group, an ethoxy group, a propoxy group, an isopropoxy group, a tert-butoxy group, a 2-ethyl-octyloxy group, a benzyloxy group, or the like.

The silyl group is, for example, but not limited to, a trimethylsilyl group, a triphenylsilyl group, or the like.

An optional substituent of L₁ and L₂ in the general formula [1] and R₁₀ to R₄₀ in the general formulae [2-1] to [2-3] may be a halogen atom, an alkyl group, an alkoxy group, an aryl group, a heterocyclic group, an amino group, or a silyl group.

The halogen atom is, for example, but not limited to, fluorine, chlorine, bromine, iodine, astatine, tennessine, or the like.

The alkyl group may be an alkyl group with 1 or more and 20 or less carbon atoms. The alkyl group is, for example, but not limited to, a methyl group, an ethyl group, a n-propyl group, an isopropyl group, a n-butyl group, a tert-butyl group, a sec-butyl group, an octyl group, a cyclohexyl group, a tert-pentyl group, a 3-methylpentan-3-yl group, a 1-adamantyl group, a 2-adamantyl group, or the like.

The alkoxy group may be an alkoxy group with 1 or more and 10 or less carbon atoms. The alkoxy group is, for example, but not limited to, a methoxy group, an ethoxy group, a propoxy group, an isopropoxy group, a tert-butoxy group, a 2-ethyl-octyloxy group, a benzyloxy group, or the like.

The aryl group may be an aryl group with 6 or more and 20 or less carbon atoms. The aryl group is, for example, but not limited to, a phenyl group, a naphthyl group, an indenyl group, a biphenyl group, a terphenyl group, a fluorenyl group, a phenanthryl group, a triphenylenyl group, a pyrenyl group, an anthranyl group, a perylenyl group, a chrysenyl group, a fluoranthenyl group, or the like.

The heterocyclic group may be a heteroaryl group with 3 or more and 20 or less carbon atoms. The heterocyclic group is, for example, but not limited to, a pyridyl group, a pyrimidyl group, a pyrazyl group, a triazyl group, a benzofuranyl group, a benzothiophenyl group, a dibenzofuranyl group, a dibenzothiophenyl group, an oxazolyl group, an oxadiazolyl group, a thiazolyl group, a thiadiazolyl group, a carbazolyl group, an acridinyl group, a phenanthrolyl group, or the like.

The silyl group is, for example, but not limited to, a trimethylsilyl group, a triphenylsilyl group, or the like.

The amino group is, for example, but not limited to, an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisolylamino group, an N-mesityl-N-phenylamino group, an N,N-dimesitylamino group, an N-phenyl-N-(4-tert-butylphenyl)amino group, an N-phenyl-N-(4-trifluoromethylphenyl)amino group, an N-piperidyl group, a carbazolyl group, an acridyl group, a trimethylamino group, a triphenylamino group, or the like.

An optional substituent of the alkyl group, the alkoxy group, the amino group, the aryl group, the heterocyclic group, the aryloxy group, and the silyl group is, for example, but not limited to, deuterium, an alkyl group, such as a methyl group, an ethyl group, a n-propyl group, an isopropyl group, a n-butyl group, or a tert-butyl group; an aralkyl group, such as a benzyl group; an aryl group, such as a phenyl group or a biphenyl group; a heterocyclic group, such as a pyridyl group or a pyrrolyl group; an amino group, such as a dimethylamino group, a diethylamino group, a dibenzylamino group, a diphenylamino group, or a ditolylamino group; an alkoxy group, such as a methoxy group, an ethoxy group, or a propoxy group; an aryloxy group, such as a phenoxy group; a halogen atom, such as fluorine, chlorine, bromine, or iodine; a cyano group, or the like.

L₁ in the general formula [1] can have one or more substituted or unsubstituted dibenzothiophenes or two or more substituted or unsubstituted xanthones.

L₂ in the general formula [1] can be represented by any one of formulae [3-1] to [3-5].

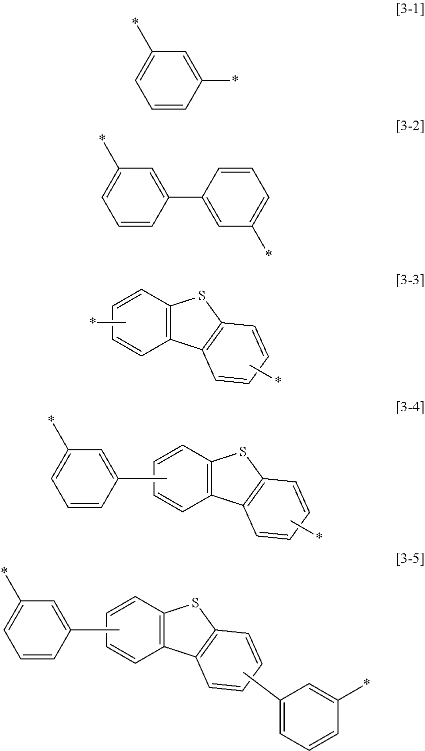

In the general formulae [3-1] to [3-5], * represents a binding position.

An organic compound according to the present disclosure has the following characteristics.
(1-1) An indolocarbazole ring with hole transport properties and a xanthone ring with electron transport properties in the molecule improve electric charges and energy accumulation and provide high durability.
(1-2) All freely rotatable bonds are composed of sp² carbons, resulting in high binding energy and improved element durability.
(1-3) An indolocarbazole ring with hole transport properties and a xanthone ring with electron transport properties are connected to each other directly or through a spacer L₁ or L₂ with a specific structure, resulting in high stability.
These characteristics are described below.
   (1-1) An Indolocarbazole Ring with Good Hole Transport Properties and High T1 or Binding Stability and a Xanthone Ring with Good Electron Transport Properties and High T1 or Binding Stability in the Molecule Improve Electric Charges and Energy Accumulation and Provide High Durability.

In inventing the organic compound represented by the general formula [1], the present inventors have paid attention to the hole transport unit, the electron transport unit, and the permanent dipole moment of the molecule.

A compound in an organic compound layer, particularly in a light-emitting layer, of an organic light-emitting element transits repeatedly between the ground state and the excited state in the process of light emission of the organic light-emitting element. In particular, in a phosphorescent organic light-emitting element, it is important to control the triplet excited state (T1), which occupies 75% of the excited state. For example, it is necessary to efficiently promote energy transfer from T1 of a host molecule to a guest molecule and to efficiently emit light from the guest molecule. Low energy transfer efficiency results in an increase in probability that generated excitation energy is used for a reaction with an adjacent molecule, and lower durability due to the generation of a quencher molecule.

It is known that the energy transfer process from T1 of a host molecule to a guest molecule occurs by Dexter energy transfer. To improve the efficiency of Dexter energy transfer, it is important to minimize the distance between a host molecule and a guest molecule.

As a result of intensive studies, the present inventors have found it effective to have one xanthone ring and at least one indolocarbazole ring to reduce the intermolecular distance between a host molecule and a guest molecule. This is characterized in that a xanthone ring and an indolocarbazole ring that suitably interact with a metal atom (for example, an iridium atom) of a guest molecule are simultaneously arranged in the molecule to increase the intermolecular interaction with the guest molecule and reduce the intermolecular distance from the guest molecule. From another perspective, a xanthone ring and an indolocarbazole ring have a different heteroatom in the skeleton and therefore characteristically have high polarization. Table 1 shows the dipole moments of two example organic compounds according to the present disclosure and a comparative organic compound (comparative compound). As shown in Table 1, the example organic compounds characteristically have a large permanent dipole moment. Furthermore, a host molecule with a large permanent dipole moment is compatible with a guest molecule with high polarity, such as an Ir complex or a Pt complex.

TABLE 1

| | | Dipole |
|---|---|---|
| | Molecular structure | moment |
| Example organic compound | 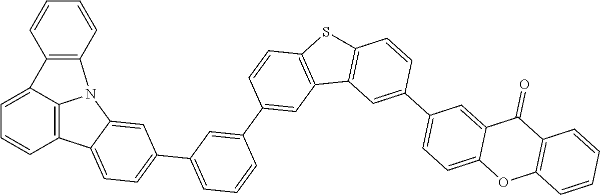 [SMILES: O=c1c2ccccc2oc2ccc(-c3ccc4sc5ccc(-c6cccc(-c7ccc8c9cccc%10c%11ccccc%11n(c8c7)c%109)c6)cc5c4c3)cc12] | 3.0 (Calc.) |
| Example organic compound | 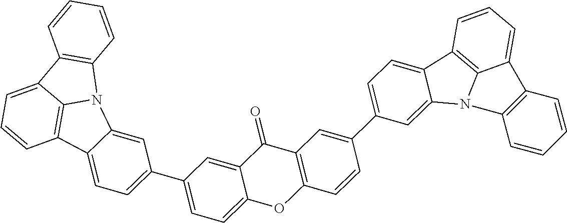 [SMILES: O=c1c2cc(-c3ccc4c5cccc6c7ccccc7n(c4c3)c65)ccc2oc2ccc(-c3ccc4c5cccc6c7ccccc7n(c4c3)c65)cc12] | 3.1 (Calc.) |
| Comparative compound | 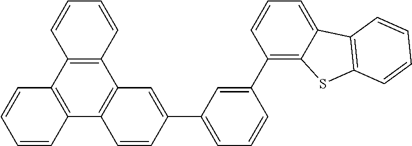 [SMILES: c1cc(-c2ccc3c4ccccc4c4ccccc4c3c2)cc(-c2cccc3c2sc2ccccc23)c1] | 0.7 (Calc.) |

### (1-2) All Freely Rotatable Bonds are Composed of Sp² Carbons, Resulting in High Binding Energy and Improved Element Durability.

A compound in an organic compound layer, particularly in a light-emitting layer, of an organic light-emitting element transits repeatedly between the ground state and the excited state in the process of light emission of the organic light-emitting element. In this process, intense motions, such as expansion and contraction and rotation, of molecules occur. At this time, an easily dissociable bond, if present, may be cleaved, and the compound may be partially separated. When the compound is partially separated, the compound has a changed structure. Thus, an easily separable compound has low durability. When such a compound is used for an organic light-emitting element, the separated portion acts as a quencher and reduces the durability of the element. Thus, a molecule with a structure without an easily dissociable bond and resistant to separation has high durability.

Having freely rotatable bonds entirely composed of sp² carbons, an organic compound according to the present disclosure is less likely to separate due to bond cleavage and has high durability. Thus, when an organic compound according to the present disclosure is used for an organic compound layer of an organic light-emitting element, the compound is rarely separated by bond cleavage during the operation of the element. Thus, the organic light-emitting element is less likely to degrade even during a long-term operation and can have high durability.

(1-3) An Indolocarbazole Ring with Hole Transport Properties and a Xanthone Ring with Electron Transport Properties are Connected to Each Other Directly or Through a Spacer L₁ or L₂ with a Specific Structure, Resulting in High Stability.

It is generally known that a compound with a combined structure of a hole transport unit and an electron transport unit undergoes charge transfer between the highest occupied molecular orbital (HOMO) and the lowest unoccupied molecular orbital (LUMO). In this case, a linking group connecting these units can have a structure resistant to electric charges. Furthermore, direct bonding without a linking group has no rotatable site, is resistant to electric charges, and has high stability.

The structure resistant to electric charges may be a benzene ring, a dibenzothiophene ring, or the like. A dibenzofuran ring, which is considered to have a structure similar to that of a dibenzothiophene ring, is considered to have a weak portion against electric charges and have low repetition stability against oxidation-reduction. When a dibenzothiophene ring and a dibenzofuran ring are considered in light of the HSAB principle, the dibenzothiophene ring suitably interacts with a metal atom (for example, an iridium atom) of a guest molecule. According to the HSAB principle, a soft acid tends to bind to a soft base, while a hard acid tends to bind to a hard base. The general characteristics of soft acids and bases are a large central atom, low electronegativity, high polarizability, and a low charge density, while hard acids and bases have the opposite characteristics. Thus, when a dibenzothiophene ring and a dibenzofuran ring are compared, the dibenzothiophene ring is classified as being soft, and the dibenzofuran ring is classified as being hard. An iridium complex used as a guest molecule has a large metal element at the center and is classified as being soft, and therefore has higher compatibility with a dibenzothiophene ring.

In terms of the molecular structure, as shown in Table 2, a dibenzothiophene ring and a dibenzofuran ring are different in the degree of distortion of the structure. The more natural angle of the angle a and the angle b in Table 2 are 120 degrees due to a bond by the sp² hybrid orbital of the carbon atom. The values in Table 2 show low stability due to a dibenzofuran ring with a more distorted structure. Thus, a dibenzothiophene ring can be used in the molecular structure.

TABLE 2

| | Angle a | Angle b |
|---|---|---|
| 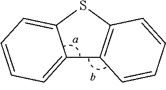 [SMILES: c1ccc2c(c1)sc1ccccc12] | 112.2° | 129.1° |
| 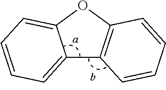 [SMILES: c1ccc2c(c1)oc1ccccc12] | 105.4° | 135.7° |

Furthermore, an organic compound according to the present disclosure can have the following characteristics.
(1-4) An indolocarbazole ring bound at a meta position of the nitrogen atom can maintain high T1 and improve the element efficiency and element durability.
(1-5) Having L₁ with a molecular weight equal to or higher than that of a biphenylene group or a dibenzothiophenylene group results in high film stability.

These characteristics are described below.

### (1-4) An Indolocarbazole Ring Bound at a Meta Position of the Nitrogen Atom can Maintain High Singlet Excited State (S1) and Improve the Element Efficiency and Element Durability.

An organic compound according to the present disclosure has a structure with an indolocarbazole ring, and the bond to the spacer L₁ is a meta position bond, not a para position or an ortho position, of the nitrogen atom. Due to the meta position bond, S1 is not lowered as compared with the para position bond or the ortho position bond, and high S1 can be maintained. Furthermore, the bond is less distorted than the ortho position bond. Thus, the binding position of the indolocarbazole ring to the spacer L₁ can be a meta position with respect to the nitrogen atom. For example, as shown in Table 3, an organic compound according to the present disclosure having the indolocarbazole ring with the meta position bond has higher S1 than Comparative Compound 1-a and Comparative Compound 1-c. Higher S1 results in higher energy transfer efficiency to a guest molecule, improved element efficiency, shorter exciton lifetime, and improved element durability. Furthermore, the dihedral angle of the indolocarbazole ring and the dibenzothiophene ring is 63.4 degrees in Comparative Compound 1-c with the ortho position bond and 38.5 degrees, which indicates higher planarity, in an organic compound according to the present disclosure. This also results in large overlap between molecules and higher efficiency of energy transfer to a guest molecule.

TABLE 3

| | | S1 | Dihedral |
|---|---|---|---|
| | Molecular structure | [nm] | angle |
| Example organic compound | 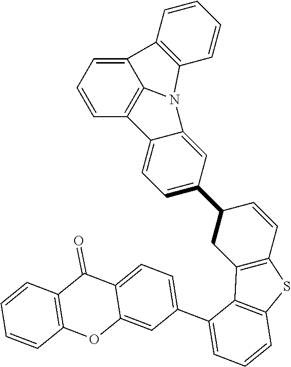 | 385 | 38.5° |
| Comparative compound 1-a | 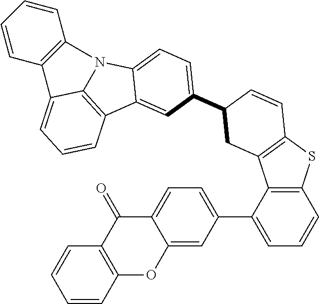 | 409 | 36.5° |
| Comparative compound 1-c | 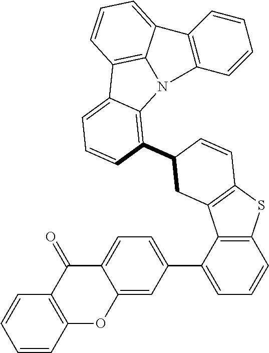 | 388 | 63.4° |

(1-5) Having L₁ with a Molecular Weight Equal to or Higher than that of a Biphenylene Group or a Dibenzothiophenylene Group Results in High Film Stability.

It is considered that a material used for an organic layer of an organic light-emitting element can typically have a higher glass transition temperature (Tg). It is known that the glass transition temperature generally tends to increase with the molecular weight. Thus, a biphenylene group or a terphenylene group is more suitable than a phenylene group in terms of the glass transition temperature. A further higher molecular weight results in a film with a more stable state.

The permanent dipole moment, T1, and S1 were calculated by molecular orbital calculation. The calculation method in the molecular orbital calculation method utilized a widely used density functional theory (DFT). B3LYP was used as the functional, and 6-31G* was used as the basis function. The molecular orbital calculation method was performed using widely used Gaussian 09 (Gaussian 09, Revision C. 01, M. J. Frisch, G. W. Trucks, H. B. Schlegel, G. E. Scuseria, M. A. Robb, J. R. Cheeseman, G. Scalmani, V. Barone, B. Mennucci, G. A. Petersson, H. Nakatsuji, M. Caricato, X. Li, H. P. Hratchian, A. F. Izmaylov, J. Bloino, G. Zheng, J. L. Sonnenberg, M. Hada, M. Ehara, K. Toyota, R. Fukuda, J. Hasegawa, M. Ishida, T. Nakajima, Y. Honda, O. Kitao, H. Nakai, T. Vreven, J. A. Montgomery, Jr., J. E. Peralta, F. Ogliaro, M. Bearpark, J. J. Heyd, E. Brothers, K. N. Kudin, V. N. Staroverov, T. Keith, R. Kobayashi, J. Normand, K. Raghavachari, A. Rendell, J. C. Burant, S. S. Iyengar, J. Tomasi, M. Cossi, N. Rega, J. M. Millam, M. Klene, J. E. Knox, J. B. Cross, V. Bakken, C. Adamo, J. Jaramillo, R. Gomperts, R. E. Stratmann, O. Yazyev, A. J. Austin, R. Cammi, C. Pomelli, J. W. Ochterski, R. L. Martin, K. Morokuma, V. G. Zakrzewski, G. A. Voth, P. Salvador, J. J. Dannenberg, S. Dapprich, A. D. Daniels, O. Farkas, J. B. Foresman, J. V. Ortiz, J. Cioslowski, and D. J. Fox, Gaussian, Inc., Wallingford CT, 2010.). This method is used for the molecular orbital calculation in the present specification.

The following are specific examples of an organic compound according to the present disclosure. However, an organic compound is not limited thereto.

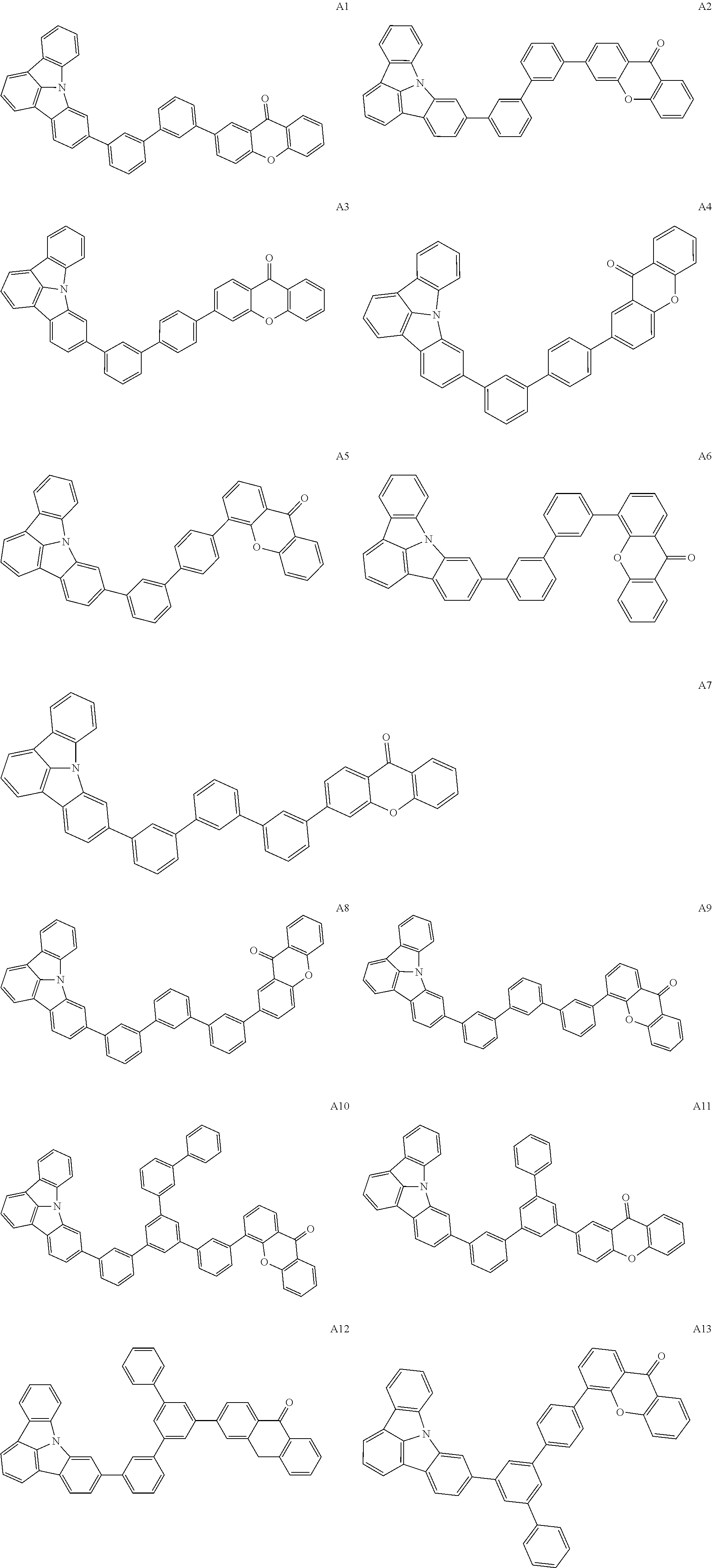

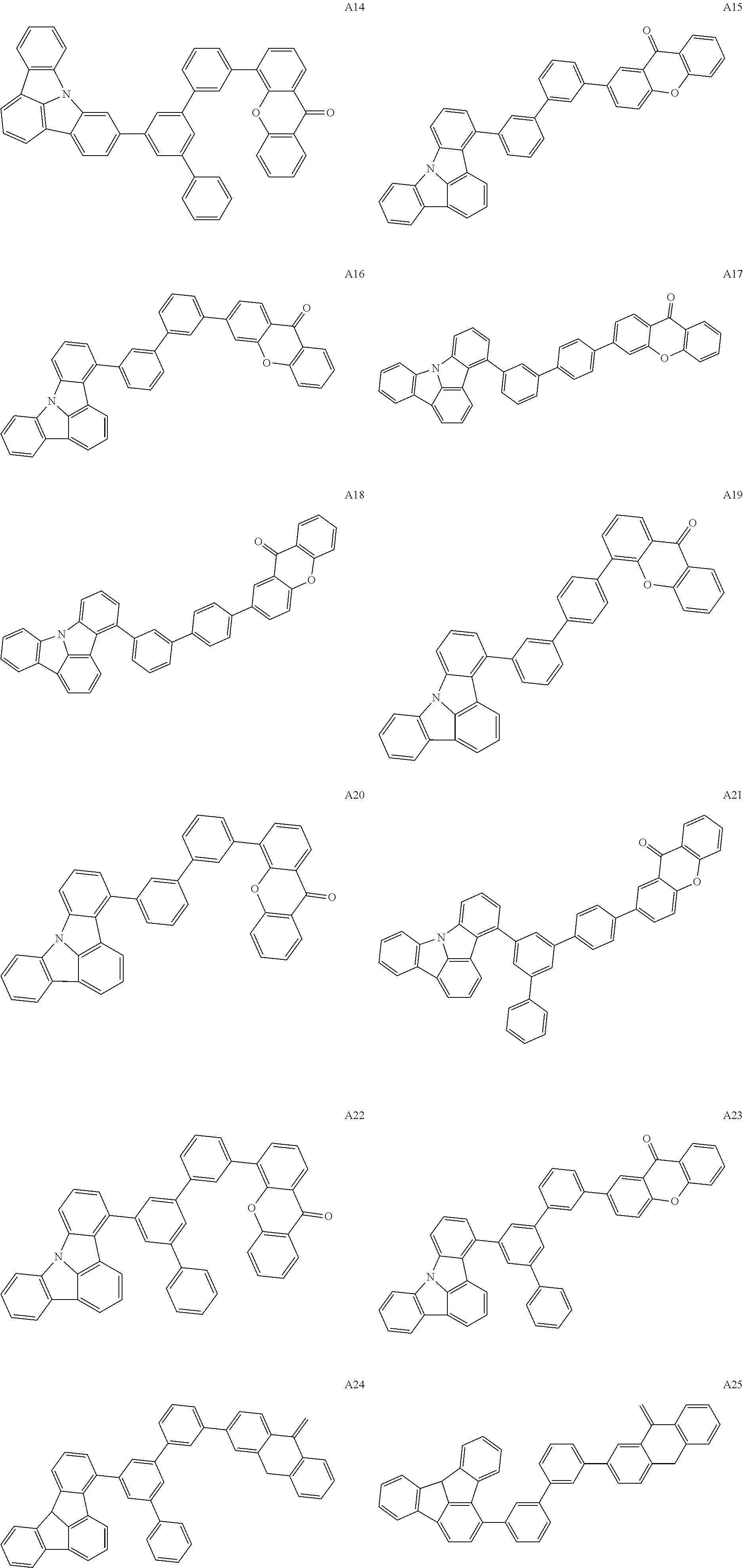

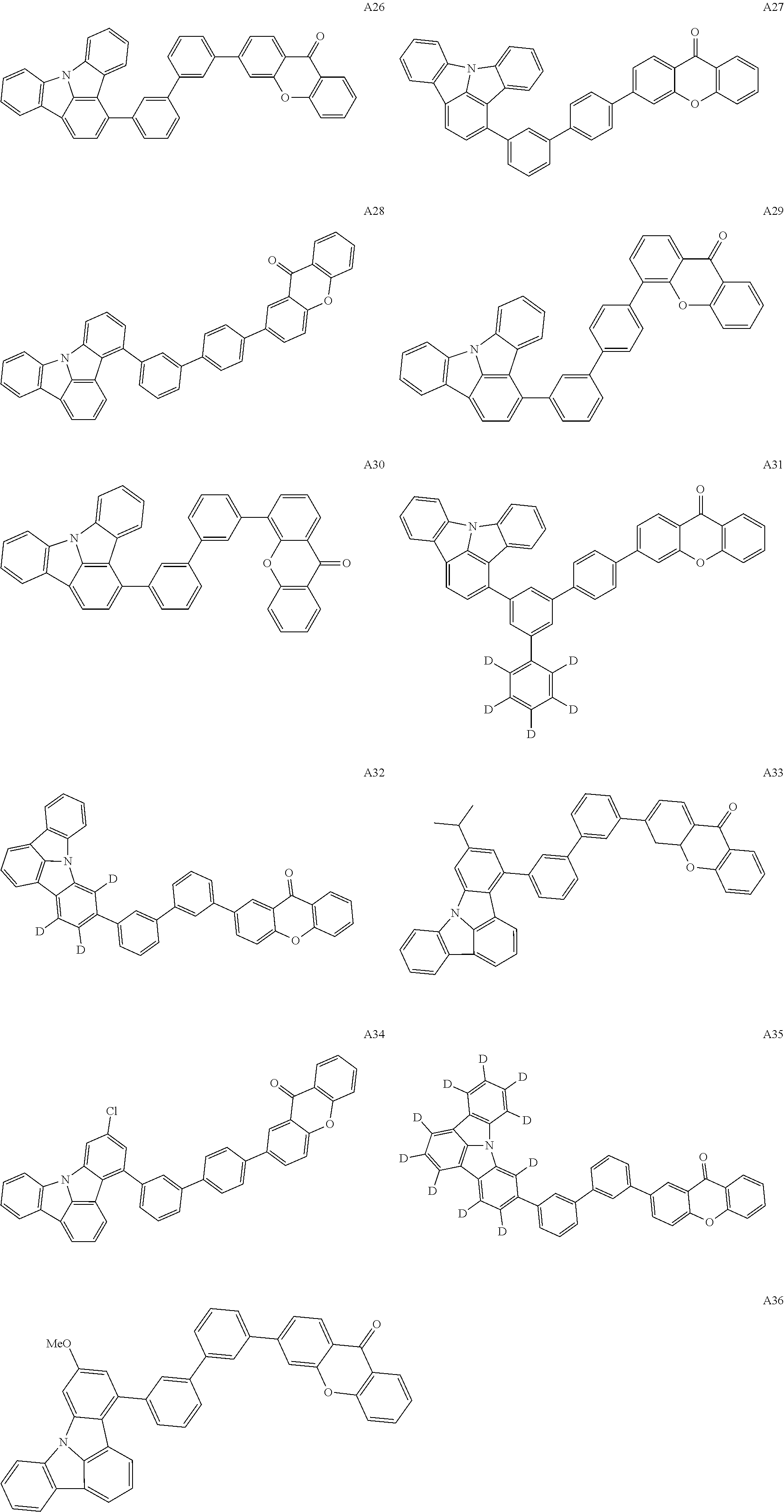

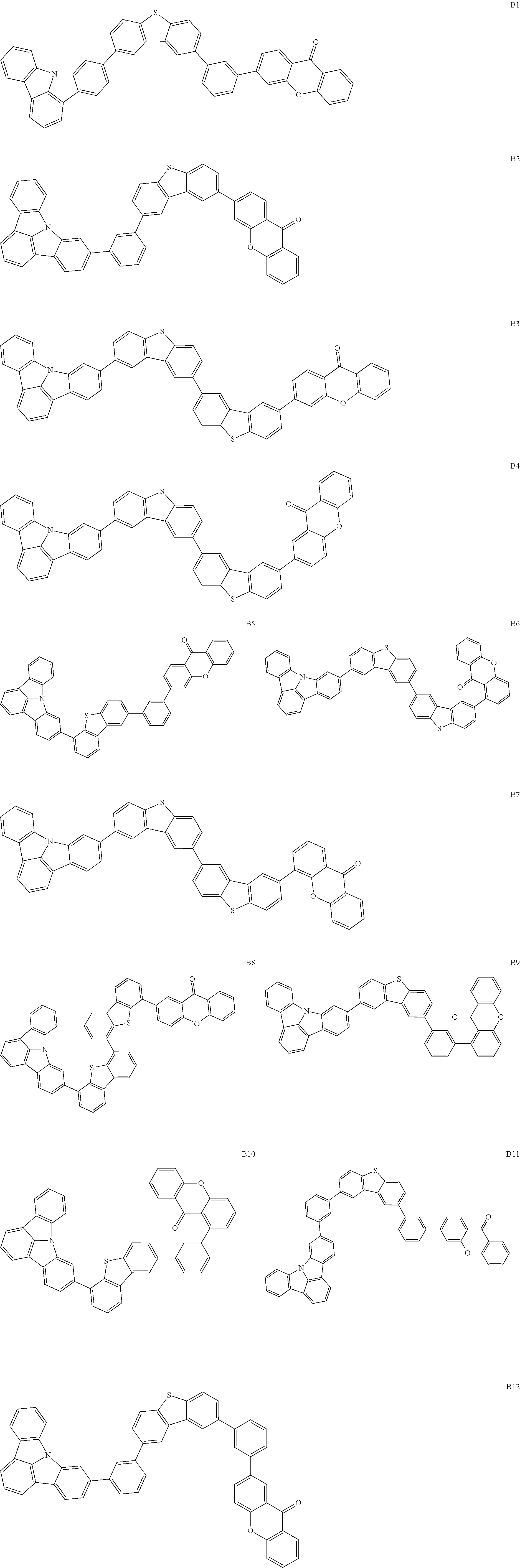

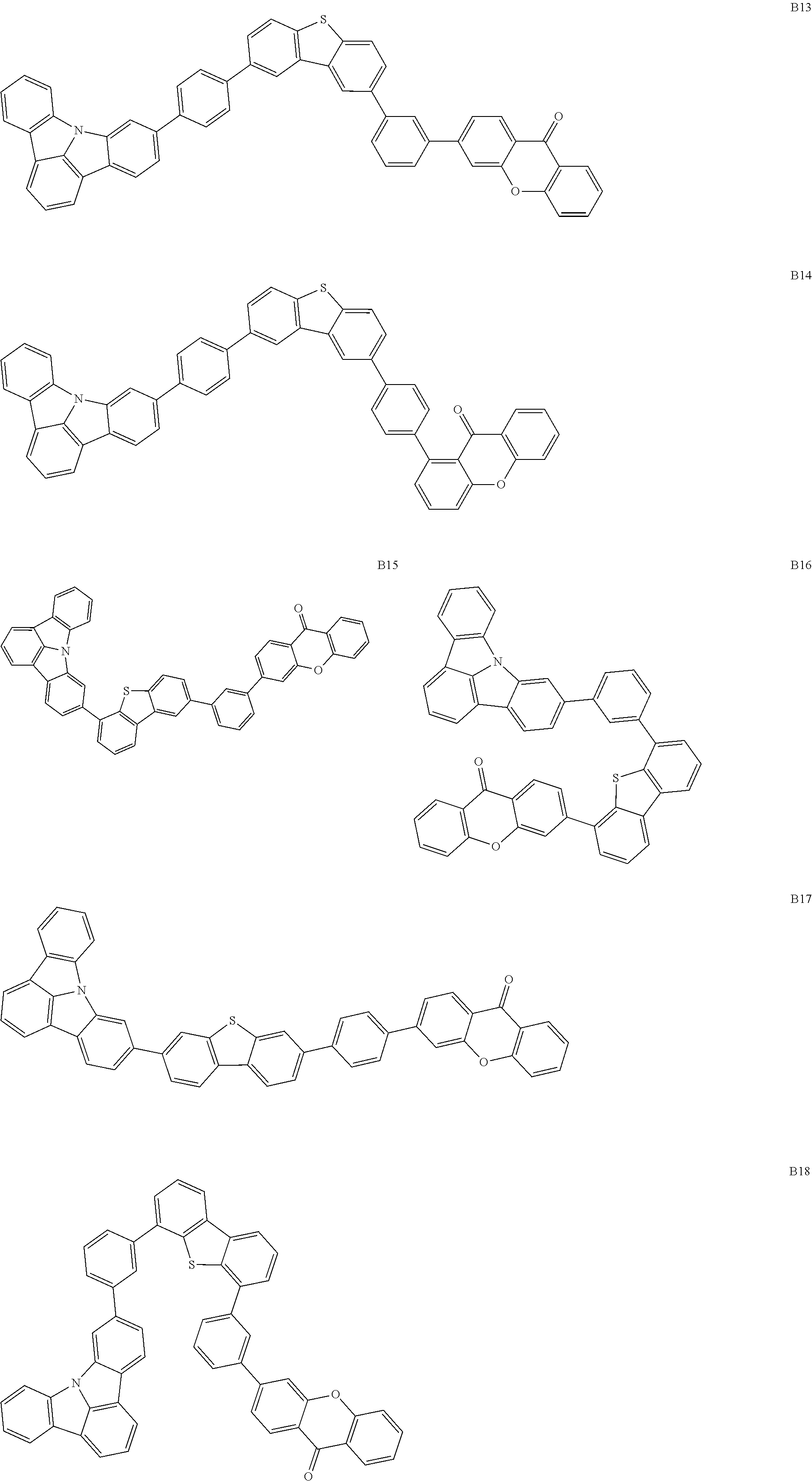

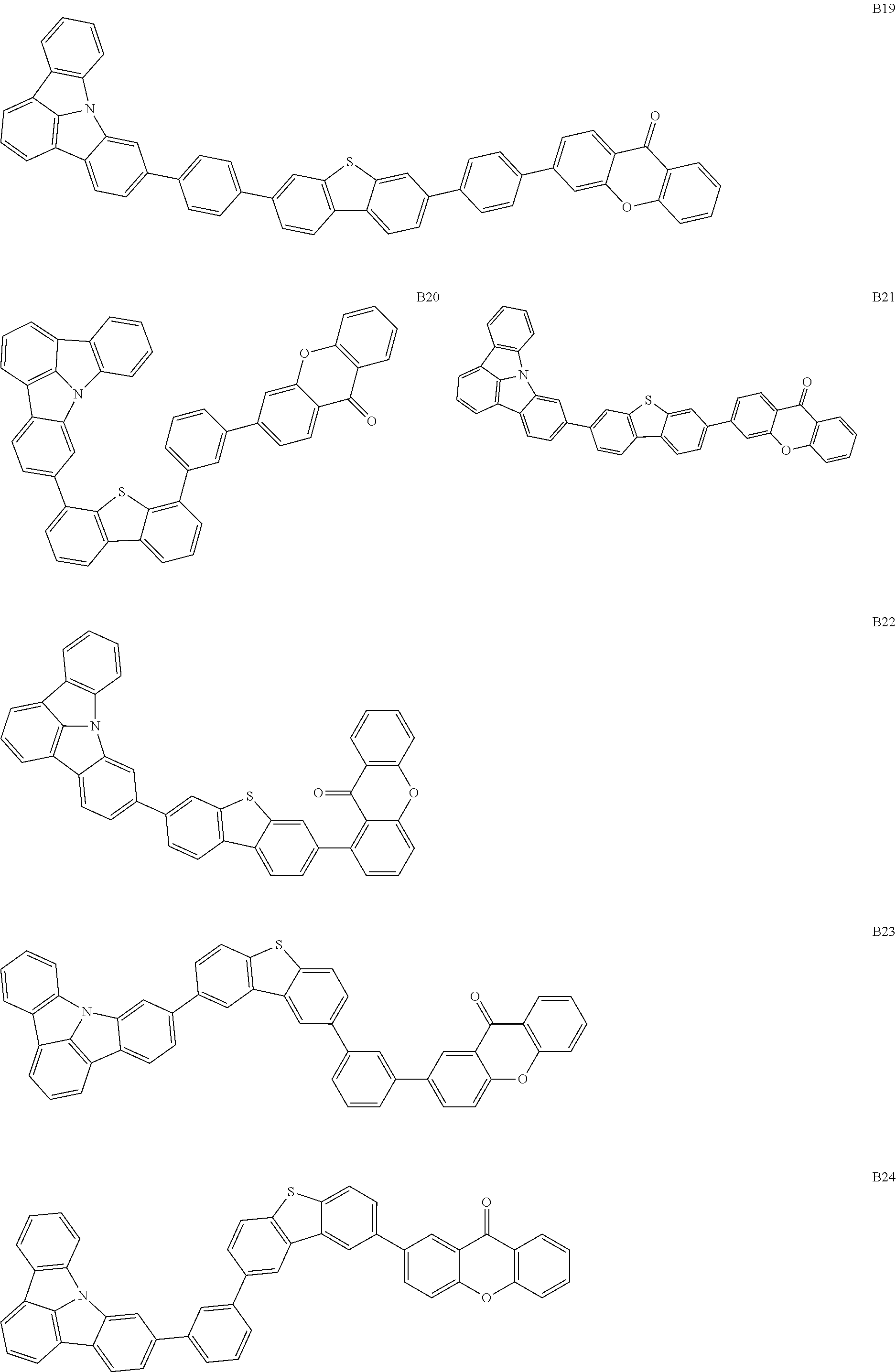

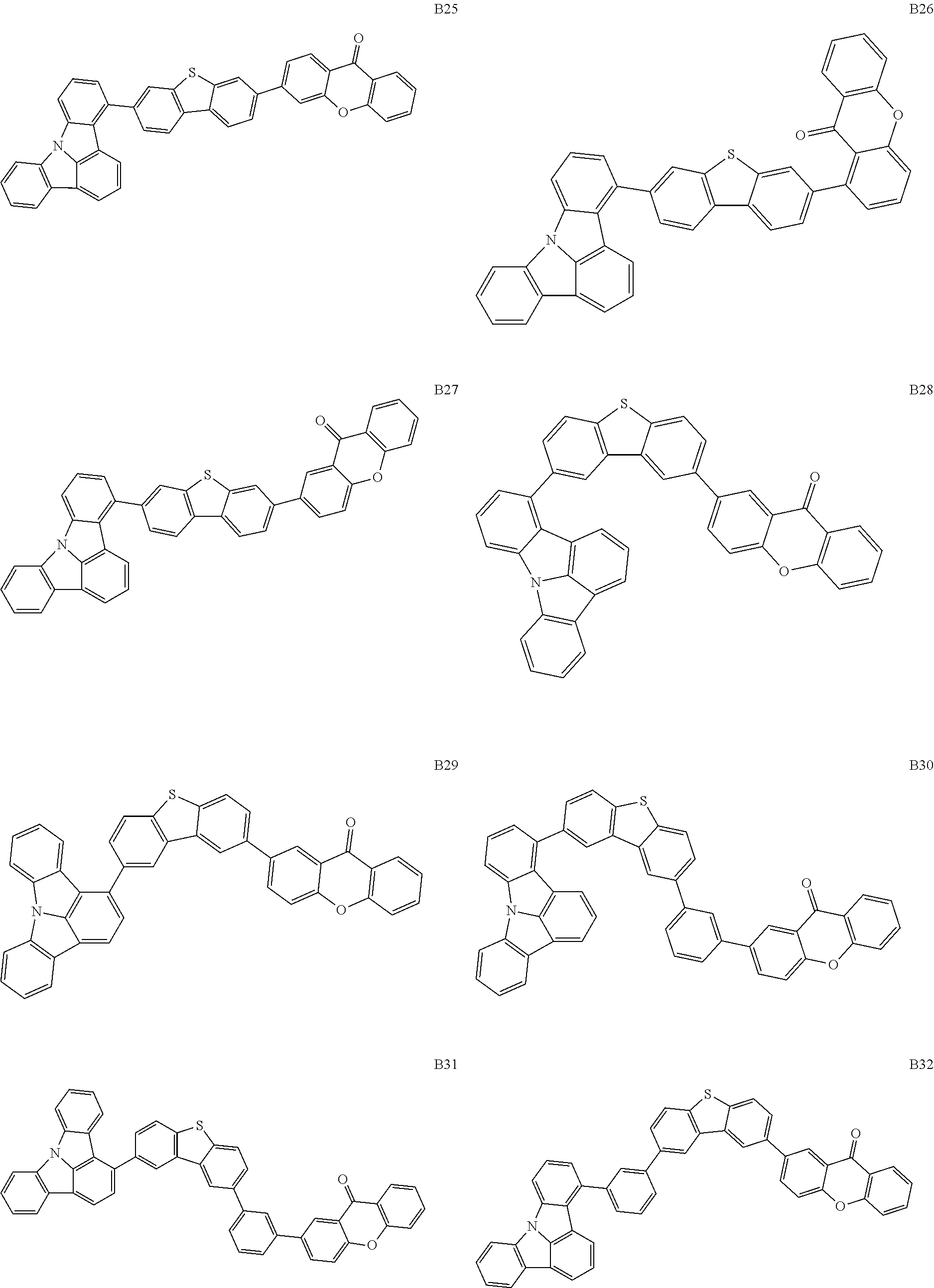

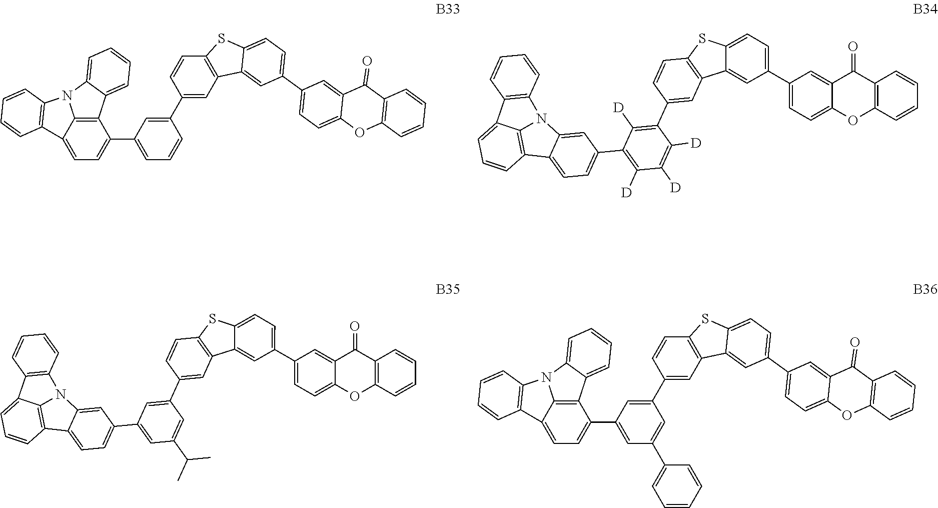 [SMILES: O=c1c2ccccc2oc2ccc(-c3ccc4sc5ccc(-c6cccc(-c7ccc8c9ccccc9n9c%10ccccc%10c7c89)c6)cc5c4c3)cc12 ; [2H]c1c([2H])c(-c2ccc3sc4ccc(-c5ccc6oc7ccccc7c(=O)c6c5)cc4c3c2)c([2H])c(-c2ccc3c4cccc5c6ccccc6n(c3c2)c54)c1[2H] ; CC(C)c1cc(-c2ccc3sc4ccc(-c5ccc6oc7ccccc7c(=O)c6c5)cc4c3c2)cc(-c2ccc3c4cccc5c6ccccc6n(c3c2)c54)c1 ; O=c1c2ccccc2oc2ccc(-c3ccc4sc5ccc(-c6cc(-c7ccccc7)cc(-c7ccc8c9ccccc9n9c%10ccccc%10c7c89)c6)cc5c4c3)cc12]

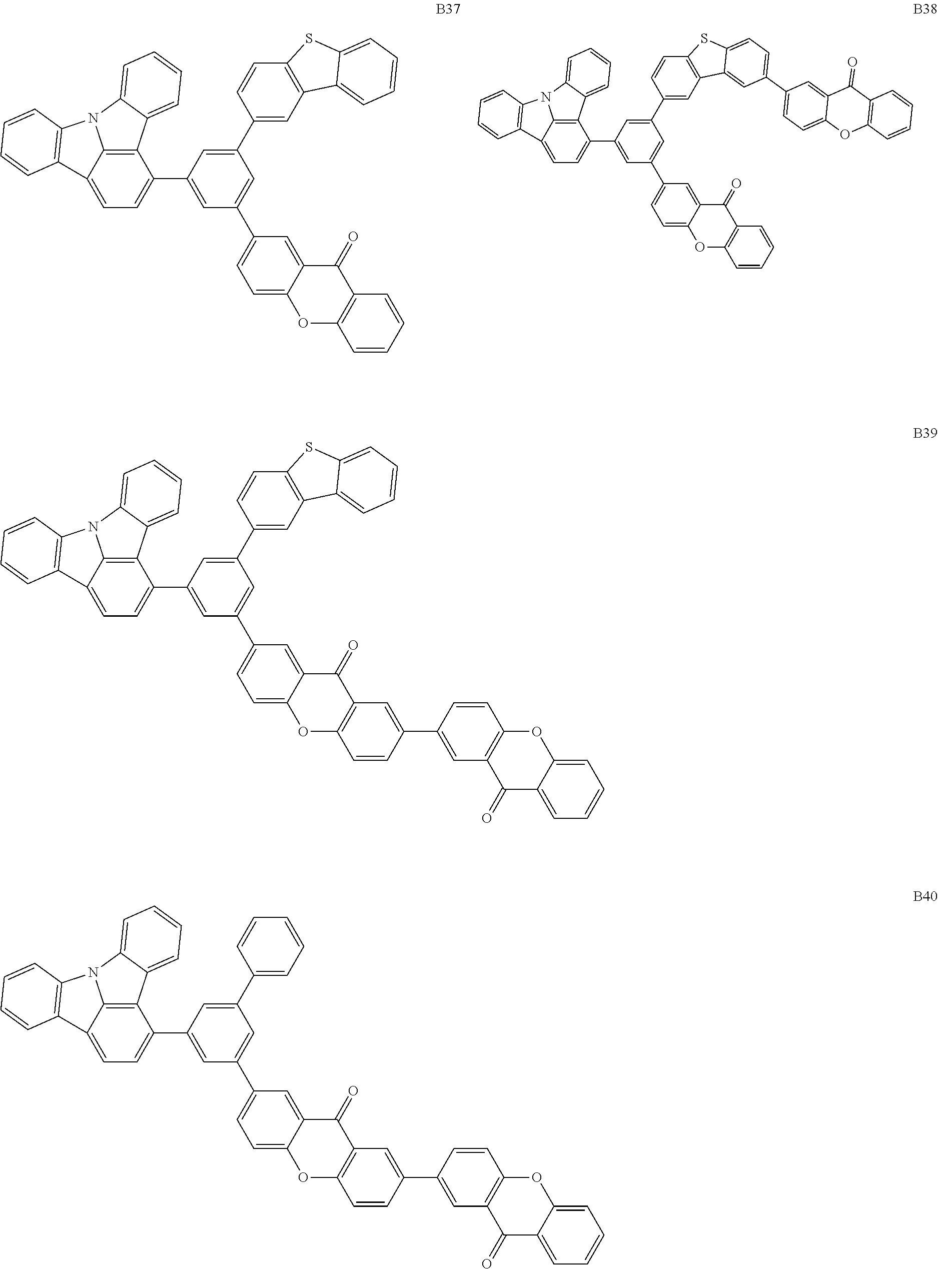 [SMILES: O=c1c2ccccc2oc2ccc(-c3cc(-c4ccc5sc6ccccc6c5c4)cc(-c4ccc5c6ccccc6n6c7ccccc7c4c56)c3)cc12 ; O=c1c2ccccc2oc2ccc(-c3cc(-c4ccc5sc6ccc(-c7ccc8oc9ccccc9c(=O)c8c7)cc6c5c4)cc(-c4ccc5c6ccccc6n6c7ccccc7c4c56)c3)cc12 ; O=c1c2ccccc2oc2ccc(-c3ccc4oc5ccc(-c6cc(-c7ccc8sc9ccccc9c8c7)cc(-c7ccc8c9ccccc9n9c%10ccccc%10c7c89)c6)cc5c(=O)c4c3)cc12 ; O=c1c2ccccc2oc2ccc(-c3ccc4oc5ccc(-c6cc(-c7ccccc7)cc(-c7ccc8c9ccccc9n9c%10ccccc%10c7c89)c6)cc5c(=O)c4c3)cc12]

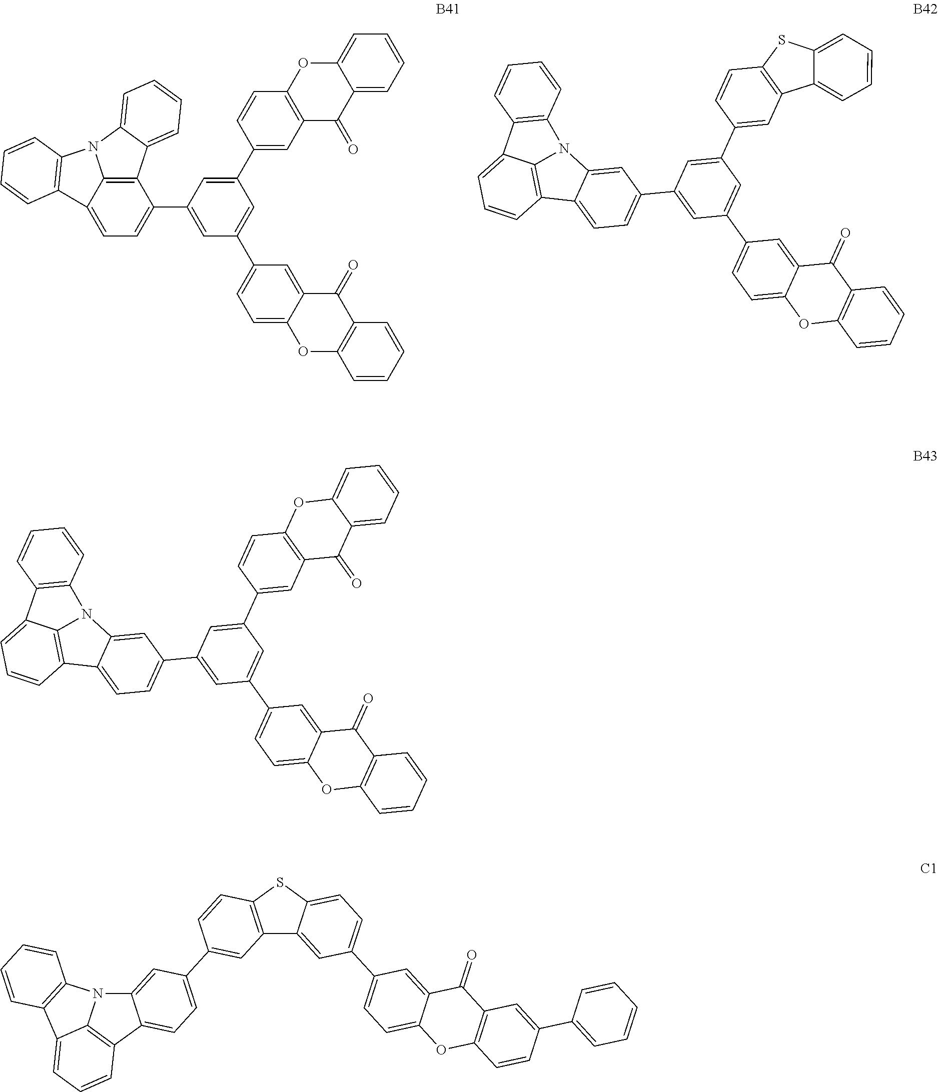 [SMILES: O=c1c2ccccc2oc2ccc(-c3cc(-c4ccc5oc6ccccc6c(=O)c5c4)cc(-c4ccc5c6ccccc6n6c7ccccc7c4c56)c3)cc12 ; O=c1c2ccccc2oc2ccc(-c3cc(-c4ccc5sc6ccccc6c5c4)cc(-c4ccc5c6cccc7c8ccccc8n(c5c4)c76)c3)cc12 ; O=c1c2ccccc2oc2ccc(-c3cc(-c4ccc5oc6ccccc6c(=O)c5c4)cc(-c4ccc5c6cccc7c8ccccc8n(c5c4)c76)c3)cc12 ; O=c1c2cc(-c3ccccc3)ccc2oc2ccc(-c3ccc4sc5ccc(-c6ccc7c8cccc9c%10ccccc%10n(c7c6)c98)cc5c4c3)cc12]

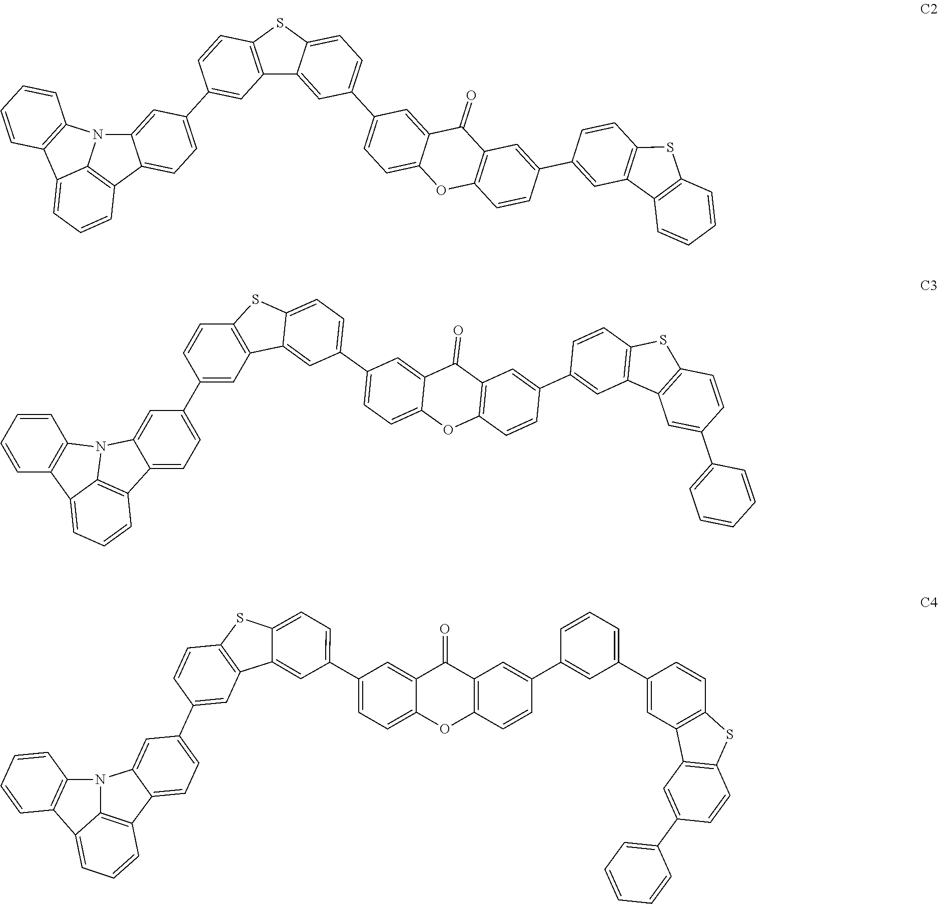 [SMILES: O=c1c2cc(-c3ccc4sc5ccccc5c4c3)ccc2oc2ccc(-c3ccc4sc5ccc(-c6ccc7c8cccc9c%10ccccc%10n(c7c6)c98)cc5c4c3)cc12 ; O=c1c2cc(-c3ccc4sc5ccc(-c6ccccc6)cc5c4c3)ccc2oc2ccc(-c3ccc4sc5ccc(-c6ccc7c8cccc9c%10ccccc%10n(c7c6)c98)cc5c4c3)cc12 ; O=c1c2cc(-c3cccc(-c4ccc5sc6ccc(-c7ccccc7)cc6c5c4)c3)ccc2oc2ccc(-c3ccc4sc5ccc(-c6ccc7c8cccc9c%10ccccc%10n(c7c6)c98)cc5c4c3)cc12]

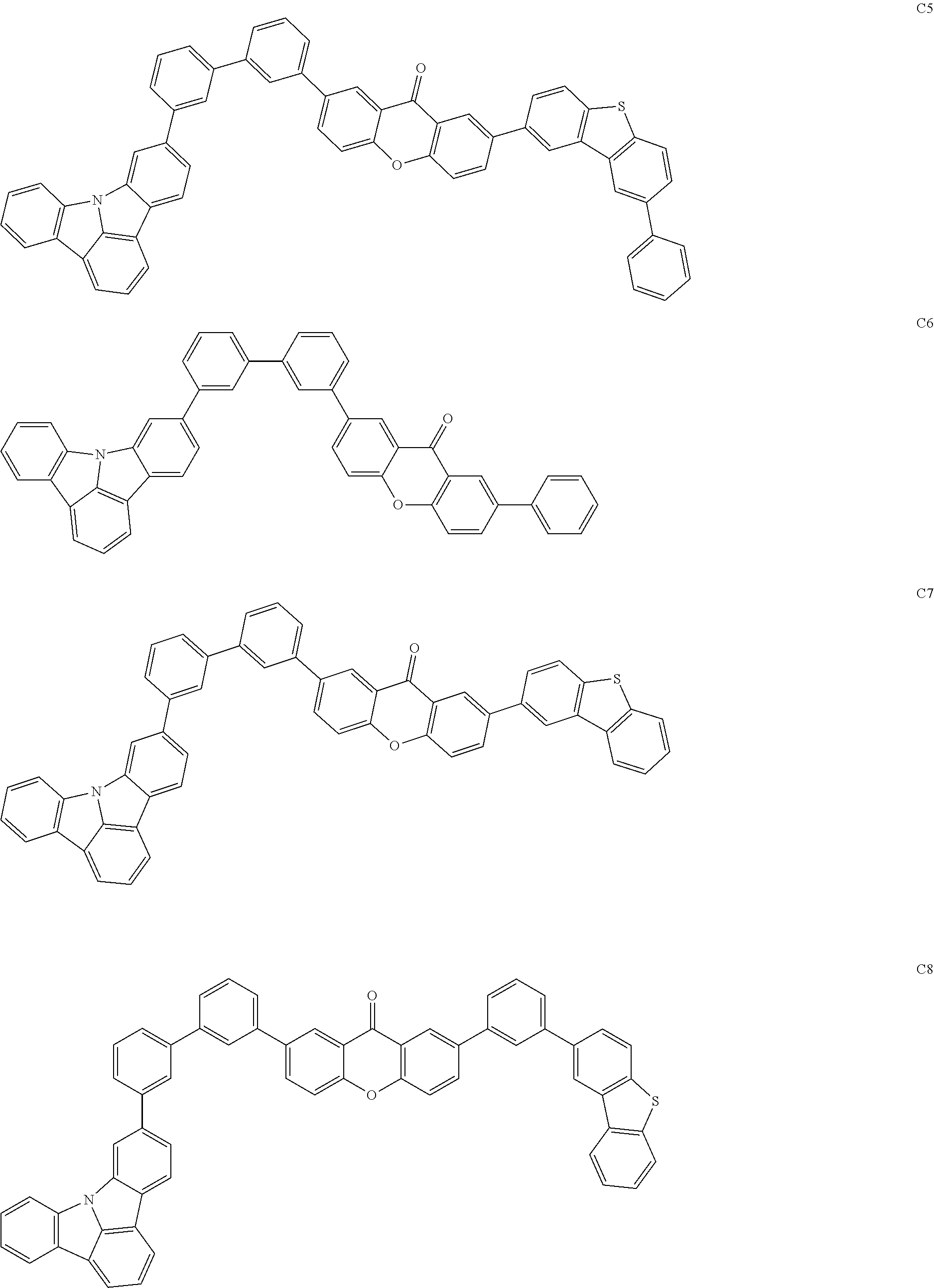 [SMILES: O=c1c2cc(-c3cccc(-c4cccc(-c5ccc6c7cccc8c9ccccc9n(c6c5)c87)c4)c3)ccc2oc2ccc(-c3ccc4sc5ccc(-c6ccccc6)cc5c4c3)cc12 ; O=c1c2cc(-c3ccccc3)ccc2oc2ccc(-c3cccc(-c4cccc(-c5ccc6c7cccc8c9ccccc9n(c6c5)c87)c4)c3)cc12 ; O=c1c2cc(-c3cccc(-c4cccc(-c5ccc6c7cccc8c9ccccc9n(c6c5)c87)c4)c3)ccc2oc2ccc(-c3ccc4sc5ccccc5c4c3)cc12 ; O=c1c2cc(-c3cccc(-c4cccc(-c5ccc6c7cccc8c9ccccc9n(c6c5)c87)c4)c3)ccc2oc2ccc(-c3cccc(-c4ccc5sc6ccccc6c5c4)c3)cc12]

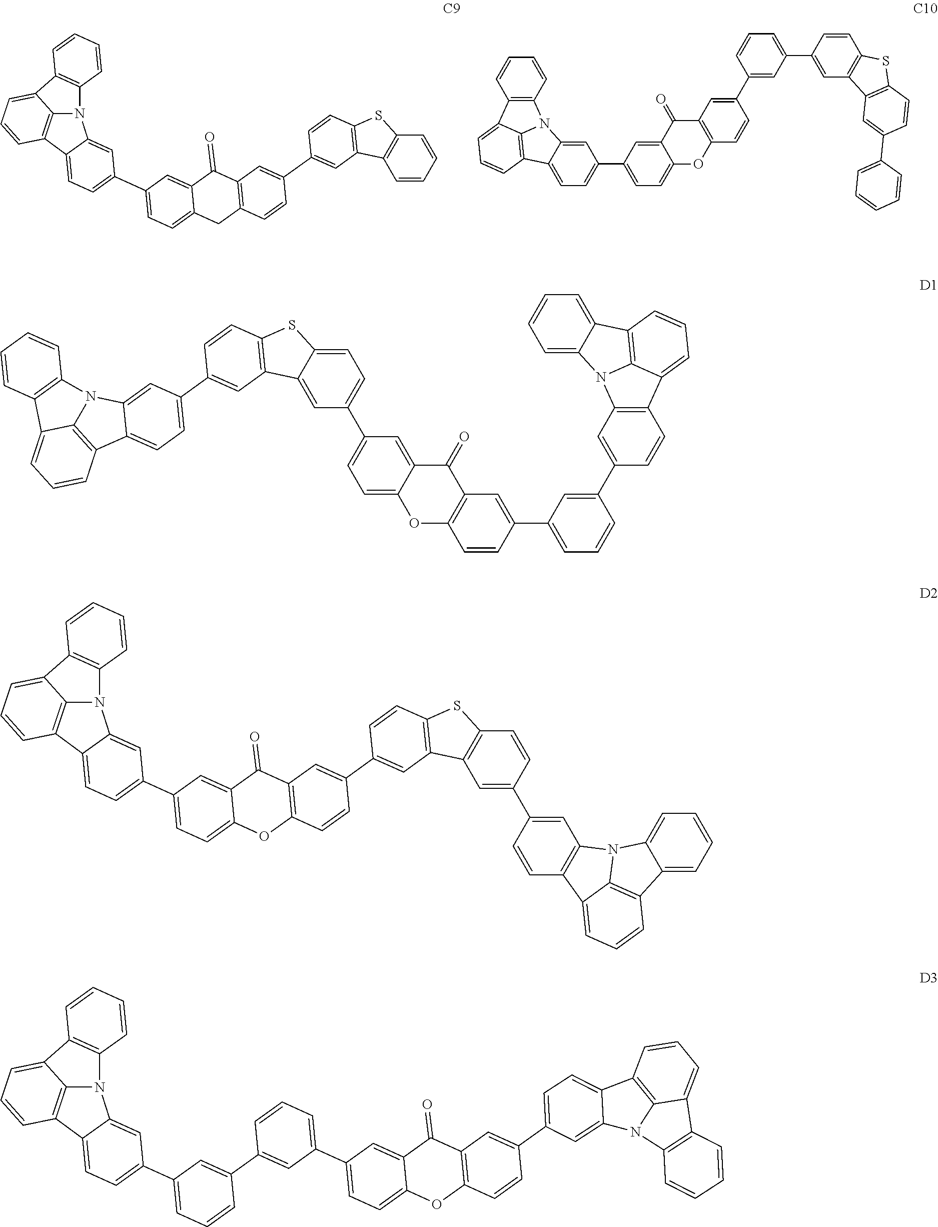

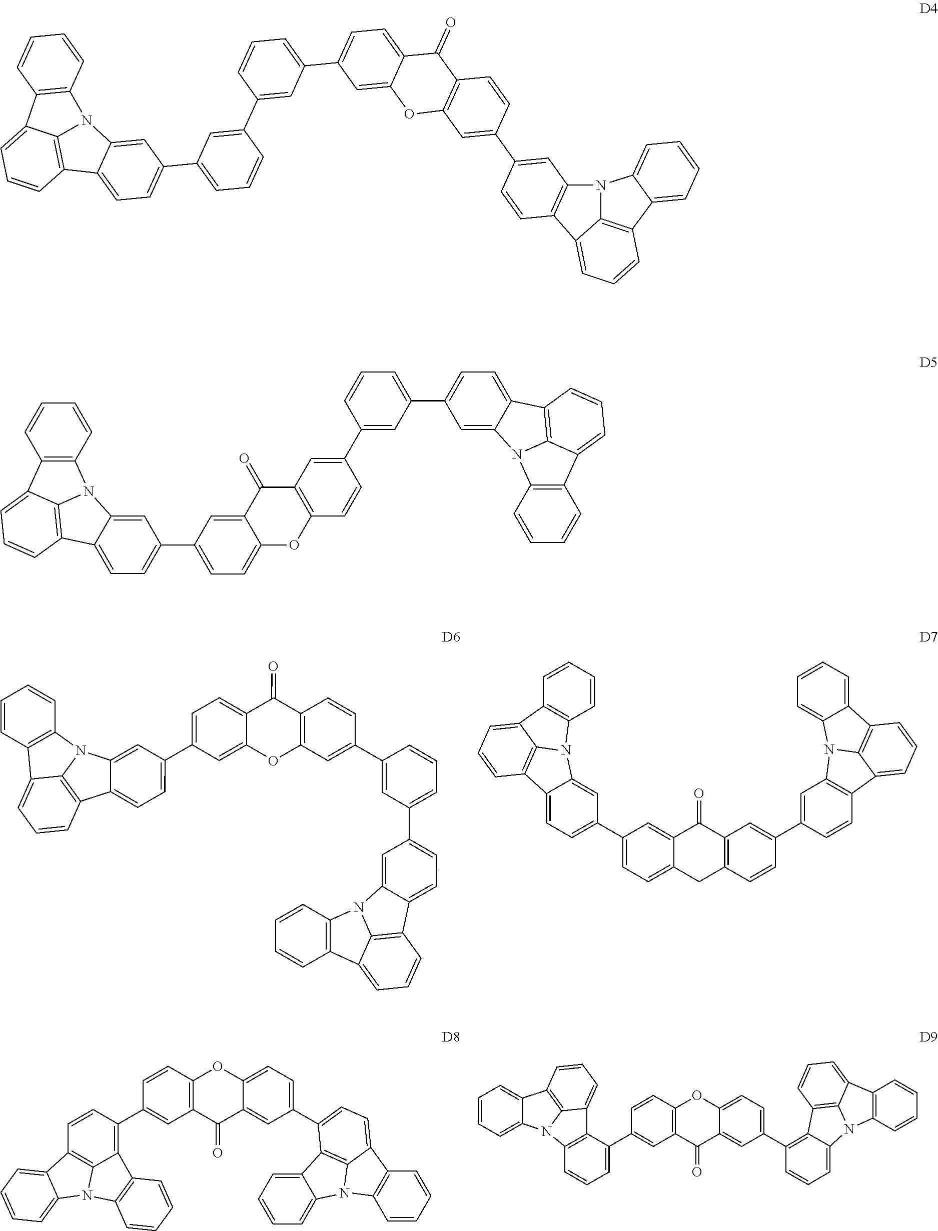

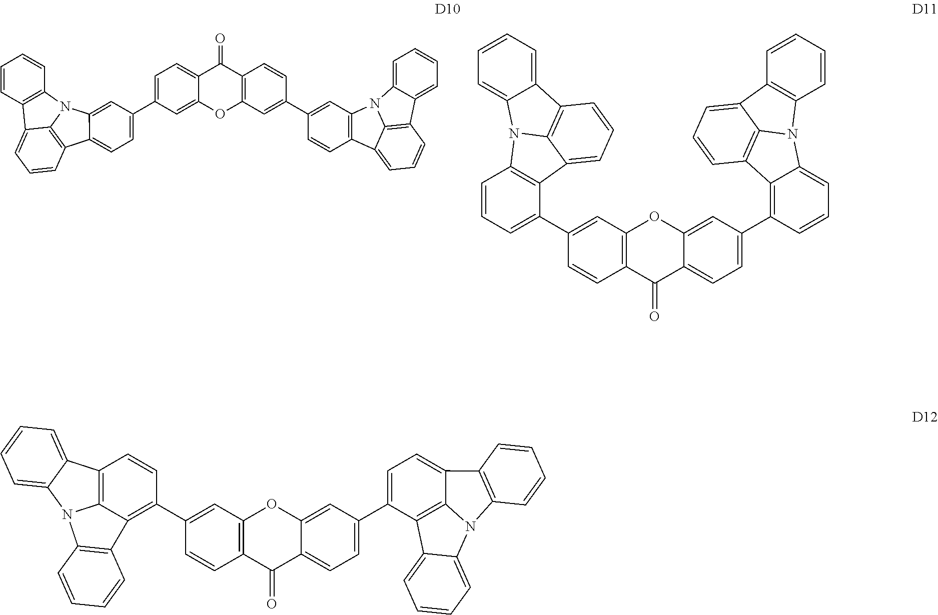

The exemplary compounds belonging to the group A are a compound group in which p=1, m and n=0, and L₁ has biphenyl or terphenyl in the general formula [1]. Among the compounds of the present disclosure, the compound group of the group A has a large number of free rotation sites, is relatively easily sublimated, and therefore has an increased margin of the sublimation temperature with respect to the decomposition temperature.

The exemplary compounds belonging to the group B are a compound group in which p=1, m and n=0, and L₁ has one or more of dibenzothiophene, xanthone, and a combination thereof in the general formula [1]. Among the compounds of the present disclosure, the compound group of the group B has three or more tricyclic or polycyclic fused-ring structures and therefore has high thermal stability. Furthermore, having a dibenzothiophene ring results in high compatibility also with a metal complex, and having two or more xanthone rings with high polarity results in high compatibility with a metal complex.

The exemplary compounds belonging to the group C are a compound group in which m=1, n=0, and L₂ has one or more benzenes, dibenzothiophenes, or combinations thereof in the formula [1]. Among the compounds of the present disclosure, the compound group of the group C has xanthone with electron transport properties located closer to the center of the molecule, has a protected lone pair, and has high stability against oxidation-reduction.

The exemplary compounds belonging to the group D are a compound group with n=1. Among the compounds of the present disclosure, the compound group of the group D has two indolocarbazole rings substituted, has high hole transport ability, and has higher hole mobility.

### [Organic Light-Emitting Element]

### <Layer Configuration>

Next, an organic light-emitting element according to the present disclosure is described. An organic light-emitting element according to the present disclosure includes at least a first electrode, a second electrode, and an organic compound layer between these electrodes, and the organic compound layer contains an organic compound according to the present disclosure. The organic compound layer is composed mainly of an organic compound and may contain an inorganic atom or an inorganic compound. For example, copper, lithium, magnesium, aluminum, iridium, platinum, molybdenum, zinc, or the like may be contained. The organic compound layer may be located between the first electrode and the second electrode and may be in contact with the first electrode and the second electrode.

One of the first electrode and the second electrode is a positive electrode, and the other is a negative electrode. In an organic light-emitting element according to the present disclosure, the organic compound layer may be a single layer or a laminate of a plurality of layers, provided that the organic compound layer has a light-emitting layer. When the organic compound layer is a laminate of a plurality of layers, the organic compound layer may have a hole injection layer, a hole transport layer, an electron-blocking layer, a hole/exciton-blocking layer, an electron transport layer, and/or an electron injection layer, in addition to a light-emitting layer. The light-emitting layer may be a single layer or a laminate of a plurality of layers.

In an organic light-emitting element according to the present disclosure, at least one layer in the organic compound layer contains an organic compound according to the present disclosure. More specifically, an organic compound according to the present disclosure is contained in any of the light-emitting layer, the hole injection layer, the hole transport layer, the electron-blocking layer, the hole/exciton-blocking layer, the electron transport layer, the electron injection layer, and the like described above. An organic compound according to the present disclosure can be contained in the hole transport layer, the electron-blocking layer, the hole/exciton-blocking layer, the electron transport layer, and/or the light-emitting layer. An organic compound according to the present disclosure can be contained in the light-emitting layer.

The light-emitting layer contains an organic compound and a light-emitting compound. The organic compound is also referred to as a host or a host material and is a compound with the highest mass ratio among the compounds constituting the light-emitting layer. The light-emitting compound is also referred to as a guest, a guest material, or a light-emitting material, is a compound that has a lower mass ratio than the host among the compounds constituting the light-emitting layer, and is a principal light-emitting compound.

An organic compound according to the present disclosure can be used as a host of the light-emitting layer. The light-emitting layer may contain a second organic compound as a second host in addition to an organic compound according to the present disclosure. The concentration of each host is preferably 10% by mass or more and 90% by mass or less, more preferably 20% by mass or more and 80% by mass or less, still more preferably 30% by mass or more and 70% by mass or less, of the entire light-emitting layer.

The concentration of the guest with respect to the host is 0.01% by mass or more and 50% by mass or less, preferably 0.1% by mass or more and 20% by mass or less, based on the total amount of the constituent materials of the light-emitting layer. From the perspective of suppressing concentration quenching, the concentration of the guest is particularly preferably 10% by mass or less.

The guest may be uniformly contained or may have a concentration gradient throughout a layer in which the host serves as a matrix. Alternatively, the guest may be partially contained in a specific region in the layer, and a light-emitting layer may have a region containing only the host and no guest.

The light-emitting layer in the present disclosure may be of monolayer or multilayer or may have a mixture of colors by containing a light-emitting material of another emission color. The term “multilayer”, as used herein, refers to a state in which a first light-emitting layer and a second light-emitting layer different from the first light-emitting layer are stacked. In such a case, the organic light-emitting element may have any emission color. More specifically, the emission color may be white or a neutral color. For a white emission color, for example, when the first light-emitting layer has a blue emission color, the second light-emitting layer has an emission color different from blue, that is, green or red. Furthermore, a third light-emitting layer that emits blue light and a charge generation layer may be provided between a single light-emitting layer or a stacked light-emitting layer and the first or second electrode. The charge generation layer has a function as a tandem element, and an electron generated from the charge generation layer and a hole injected from the first electrode are recombined and generate an exciton, and a hole generated from the charge generation layer and an electron injected from the second electrode are recombined and generate an exciton. Thus, the internal quantum efficiency is doubled. In this case, an organic light-emitting element according to the present disclosure can be applied to one side of a tandem element as a yellow light-emitting layer as a complementary color of blue light emission. Thus, a stacked light-emitting layer can be used to constitute a tandem element structure with a blue-light-emitting layer to provide a white-light-emitting element. The third light-emitting layer contains at least a third organic compound and a fourth organic compound. The third organic compound is a host material, and the fourth organic compound is a blue-light-emitting material.

A light-emitting layer according to the present disclosure can contain an electron transport compound. A phosphorescent compound exemplified by an iridium complex is often a compound with a shallow HOMO and good hole transport properties. On the contrary, shallow LUMO results in an unstable anion state and poor electron transport properties in many cases. Thus, mixing with another electron transport compound can increase the electron transport ability and form a more stable light-emitting layer.

A light-emitting layer can be formed by vapor deposition or coating.

A specific element structure of the organic light-emitting element according to the present embodiment may be a multilayer element structure including an electrode layer and an organic compound layer shown in the following (1) to (6) sequentially stacked on a substrate. In any of the element structures, the organic compound layer always includes a light-emitting layer containing a light-emitting material.
(1) positive electrode/light-emitting layer/negative electrode
(2) positive electrode/hole transport layer/light-emitting layer/electron transport layer/negative electrode
(3) positive electrode/hole transport layer/light-emitting layer/electron transport layer/electron injection layer/negative electrode
(4) positive electrode/hole injection layer/hole transport layer/light-emitting layer/electron transport layer/negative electrode
(5) positive electrode/hole injection layer/hole transport layer/light-emitting layer/electron transport layer/electron injection layer/negative electrode
(6) positive electrode/hole transport layer/electron-blocking layer/light-emitting layer/hole-blocking layer/electron transport layer/negative electrode

However, these element structure examples are only very basic element structures, and the present disclosure is not limited to these structures. Various layer structures are possible; for example, an insulating layer, an adhesive layer, or an interference layer is formed at an interface between an electrode and an organic compound layer, an electron transport layer or a hole transport layer is composed of two layers with different ionization potentials, or a light-emitting layer is composed of two layers formed of different light-emitting materials.

Among the element structures shown in (1) to (6), the structure (6) has both an electron-blocking layer and a hole-blocking layer. Thus, the electron-blocking layer and the hole-blocking layer in (6) can securely confine carriers of both holes and electrons in the light-emitting layer. Thus, the organic light-emitting element has no carrier leakage and high light emission efficiency.

Here, in an organic light-emitting element according to the present disclosure, all freely rotatable single bonds of an organic compound constituting the light-emitting layer can be carbon-carbon bonds or bonds between sp² carbons, that is, the light-emitting layer can be composed of a host material with high planarity. This results in higher hole transport ability and electron transport ability than typical organic light-emitting elements. This also results in the electron-blocking layer and the hole-blocking layer playing important roles. For example, since the hole-blocking layer needs to be stable to holes, a hole-blocking layer compound can be an organic compound with low reactivity or an organic compound composed only of a hydrocarbon. For example, since the electron-blocking layer also needs to be stable to electrons, a compound constituting the electron-blocking layer can be an organic compound with low reactivity or an organic compound in which all the freely rotatable single bonds can be carbon-carbon bonds or bonds between sp² carbons.

The mode (element form) of extracting light from the light-emitting layer may be a so-called bottom emission mode of extracting light from an electrode on the substrate side or a so-called top emission mode of extracting light from the side opposite the substrate side. The mode may also be a double-sided extraction mode of extracting light from the substrate side and from the side opposite the substrate side.

### <Constituent Material of Organic Compound Layer>

An organic compound according to the present disclosure can also be used as a constituent material of an organic compound layer other than the light-emitting layer constituting an organic light-emitting element according to the present disclosure. More specifically, an organic compound according to the present disclosure may be used as a constituent material of an electron transport layer, an electron injection layer, a hole transport layer, a hole injection layer, a hole-blocking layer, and the like. In such a case, the organic light-emitting element may have any emission color. More specifically, the emission color may be white or a neutral color.

If necessary, an organic light-emitting element according to the present disclosure may be used in combination with a known low-molecular-weight or high-molecular-weight hole injection compound or hole transport compound, host compound, light-emitting compound, electron injection compound, electron transport compound, or the like. Examples of these compounds are described below.

The hole injection/transport material can be a material with high hole mobility to facilitate the injection of a hole from a positive electrode and to transport the injected hole to a light-emitting layer. Furthermore, a material with a high glass transition temperature can be used to suppress degradation of film quality, such as crystallization, in an organic light-emitting element. Examples of a low-molecular-weight or high-molecular-weight material with hole injection/transport ability include, but are not limited to, a triarylamine derivative, an aryl carbazole derivative, a phenylenediamine derivative, a stilbene derivative, a phthalocyanine derivative, a porphyrin derivative, polyvinylcarbazole, polythiophene, and another electrically conductive polymer. Furthermore, the hole injection/transport material can also be suitable for use in an electron-blocking layer. Specific examples of a compound that can be used as the hole injection/transport material include, but are not limited to, the following.

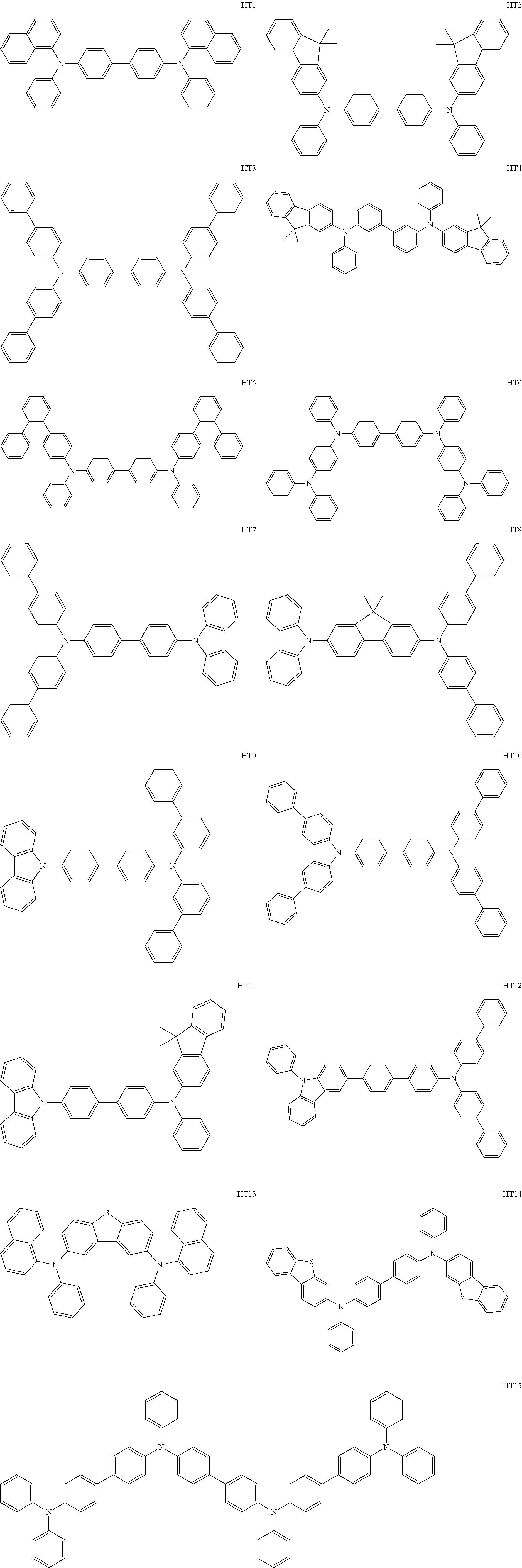

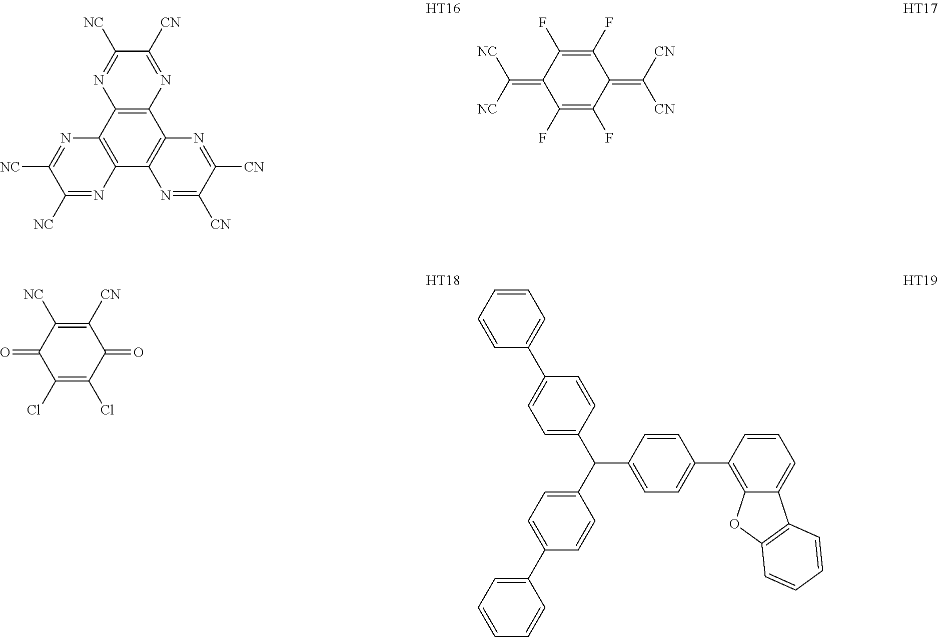 [SMILES: N#Cc1nc2c3nc(C#N)c(C#N)nc3c3nc(C#N)c(C#N)nc3c2nc1C#N ; N#CC(C#N)=c1c(F)c(F)c(=C(C#N)C#N)c(F)c1F ; N#CC1=C(C#N)C(=O)C(Cl)=C(Cl)C1=O ; c1ccc(-c2ccc(C(c3ccc(-c4ccccc4)cc3)c3ccc(-c4cccc5c4oc4ccccc45)cc3)cc2)cc1]

A light-emitting material mainly related to the light-emitting function may be, in addition to an organometallic complex, a fused-ring compound (for example, a fluorene derivative, a naphthalene derivative, a pyrene derivative, a perylene derivative, a tetracene derivative, an anthracene derivative, rubrene, or the like), a quinacridone derivative, a coumarin derivative, a stilbene derivative, an organoaluminum complex, such as tris(8-quinolinolato)aluminum, an iridium complex, a platinum complex, a rhenium complex, a copper complex, an europium complex, a ruthenium complex, or a polymer derivative, such as a poly(phenylene vinylene) derivative, a polyfluorene derivative, or a polyphenylene derivative. Specific examples of a compound that can be used as a light-emitting material include, but are not limited to, the following.

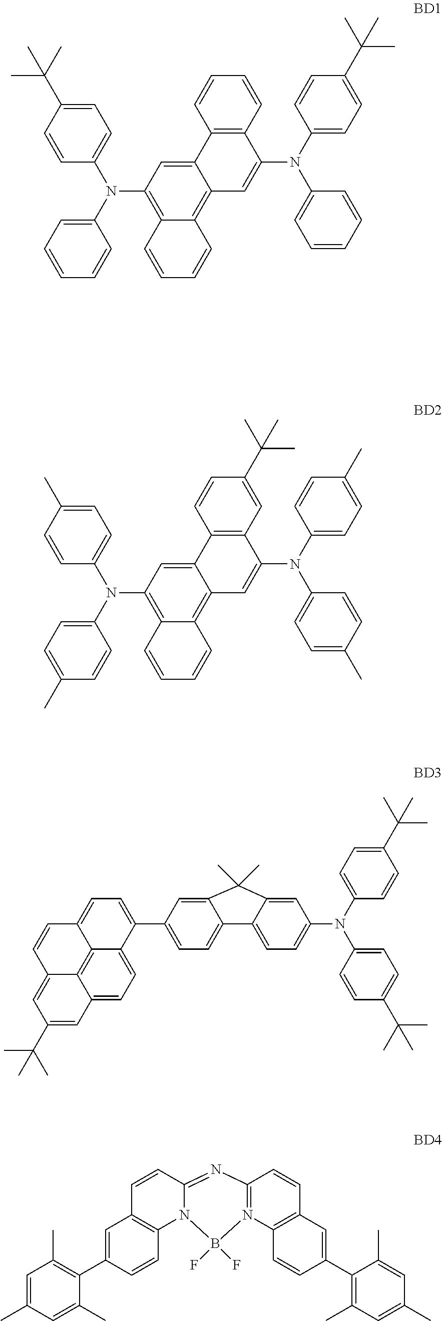

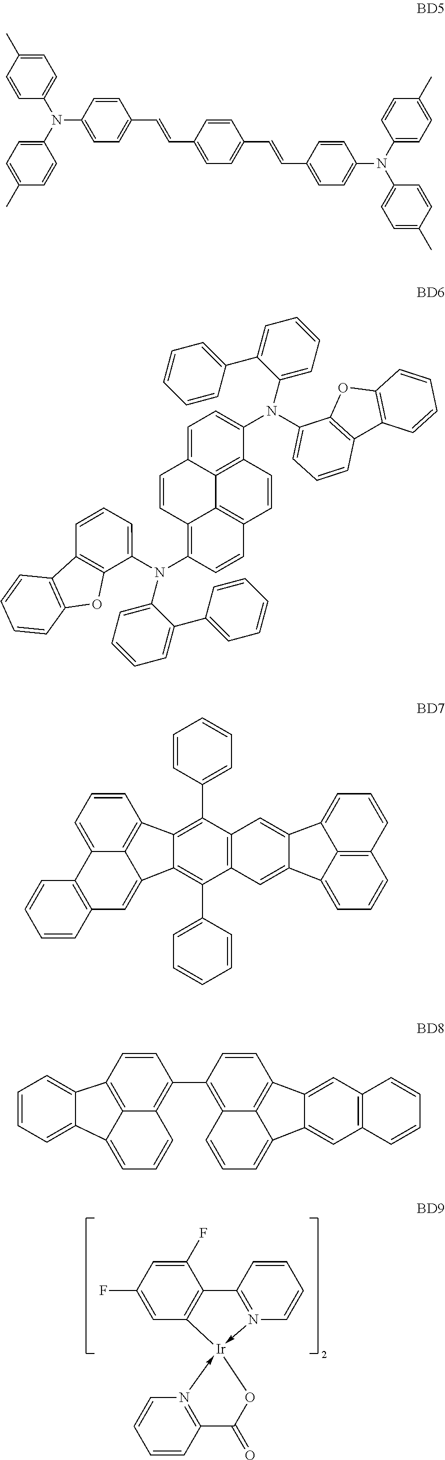

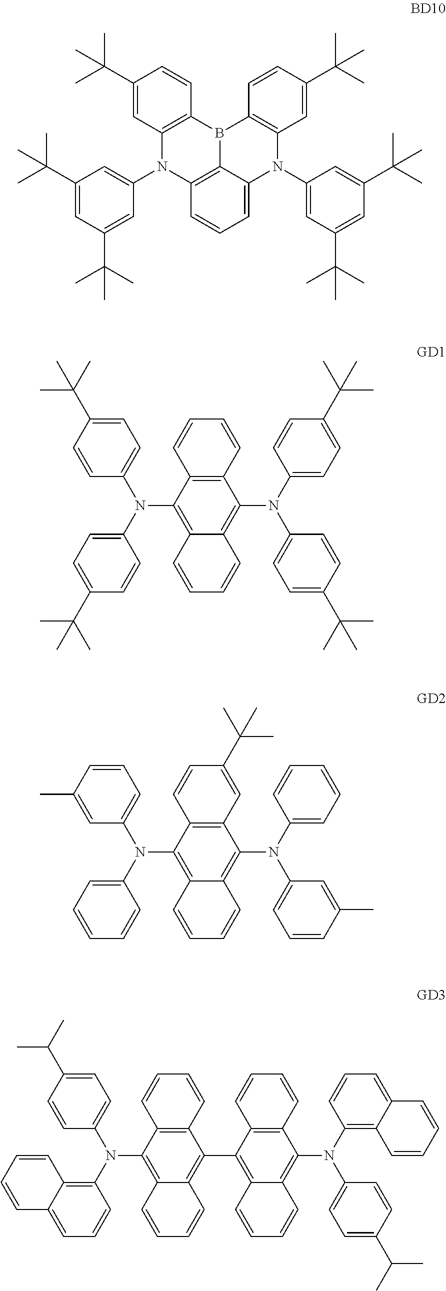

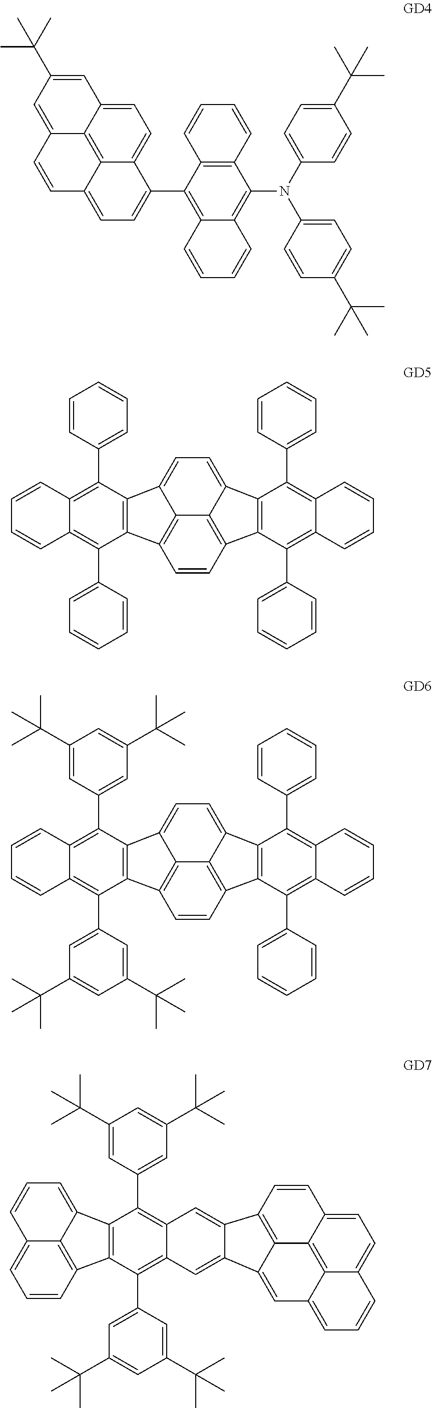

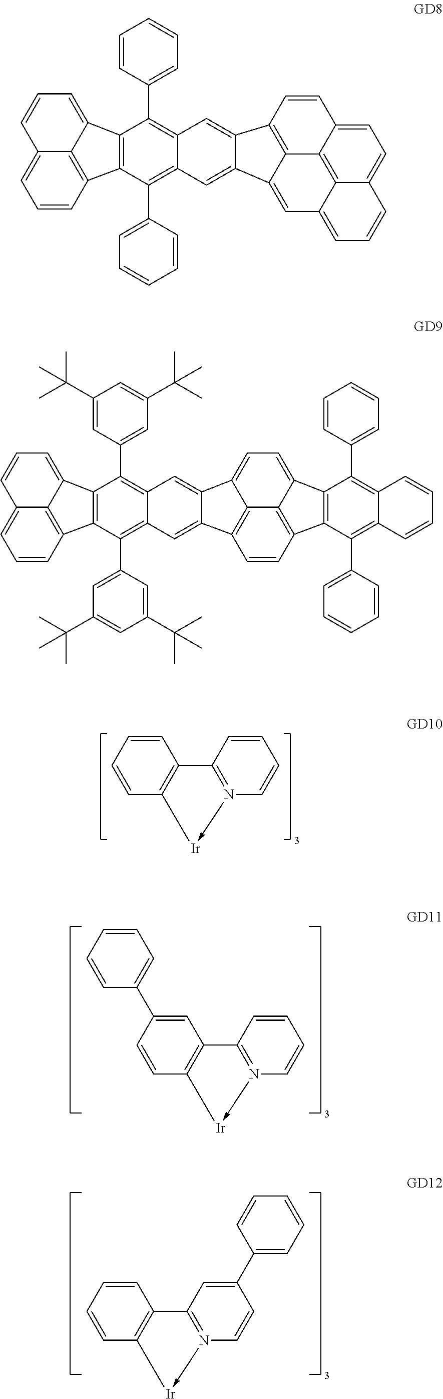

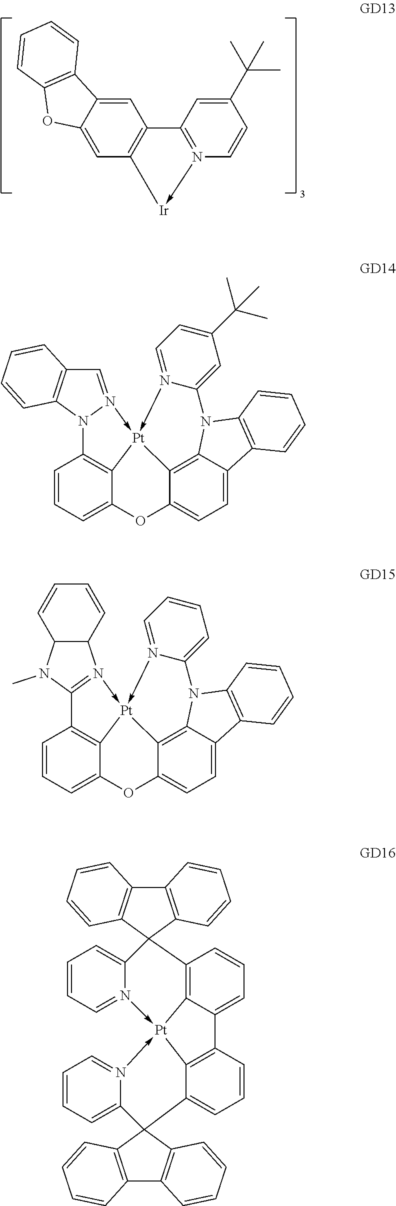

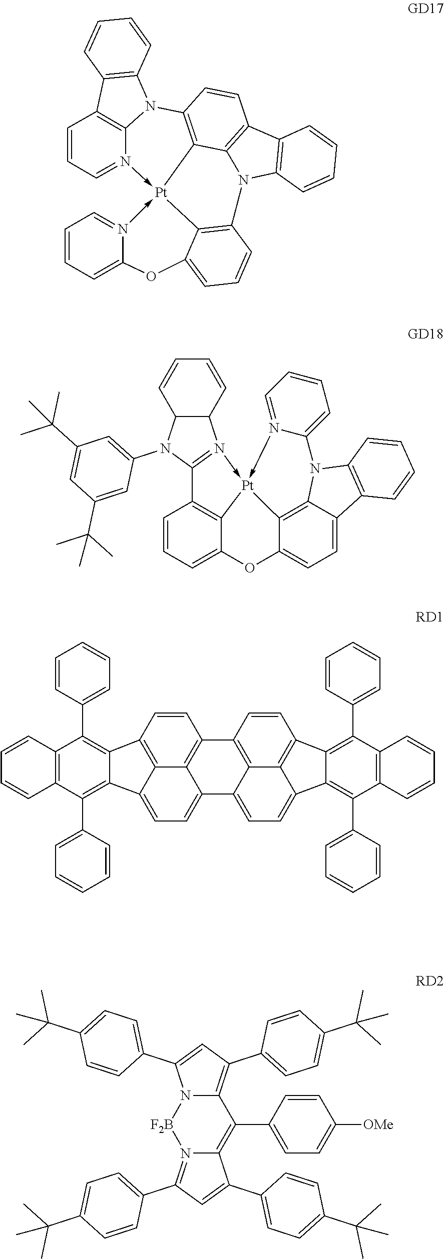

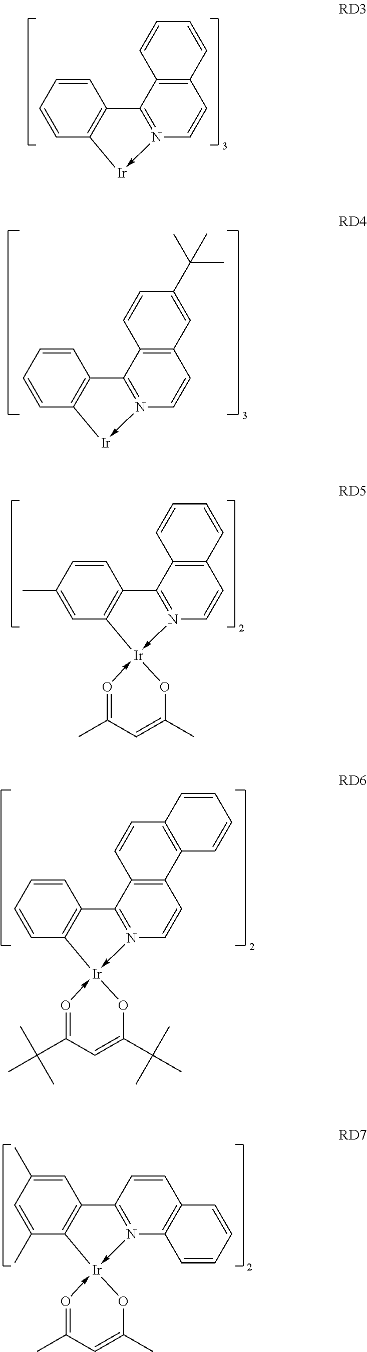

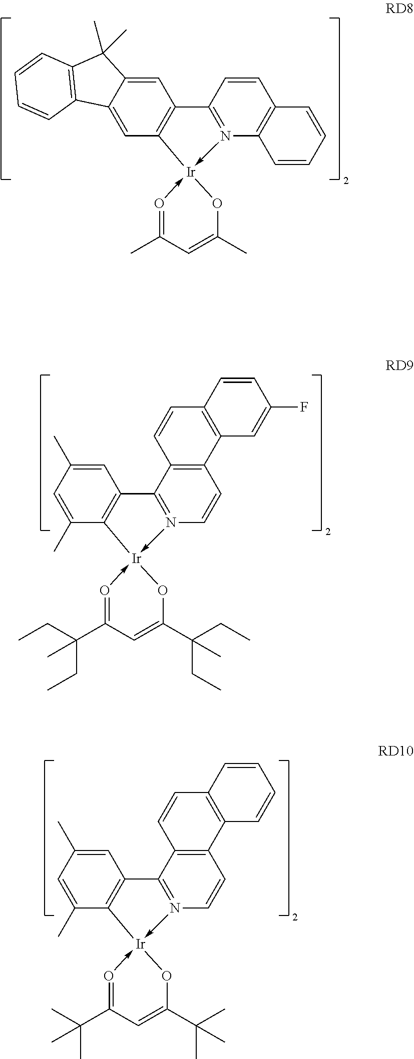

A compound other than an organic compound according to the present disclosure may be contained as a third component serving as a host or assist material contained in a light-emitting layer. The third component is, for example, an aromatic hydrocarbon compound or a derivative thereof, a carbazole derivative, an azine derivative, a xanthone derivative, a dibenzofuran derivative, a dibenzothiophene derivative, an organoaluminum complex, such as tris(8-quinolinolato)aluminum, an organoberyllium complex, or the like.

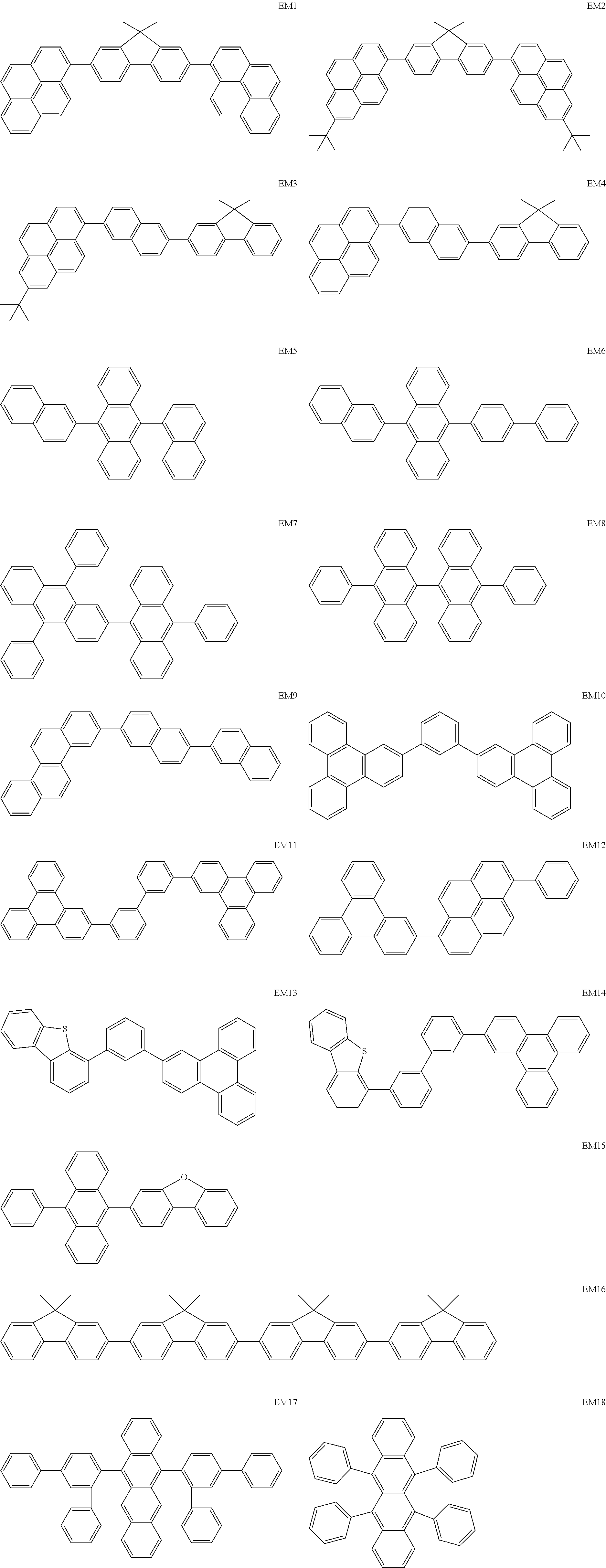

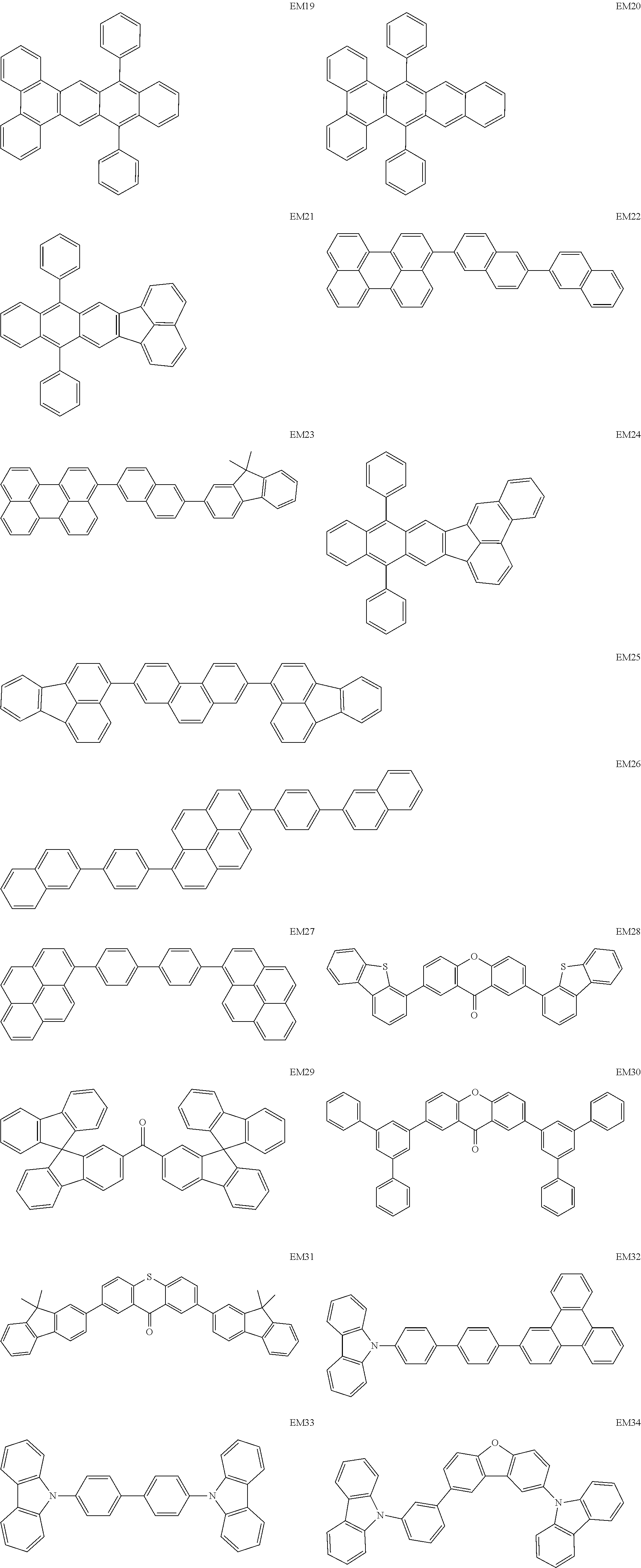

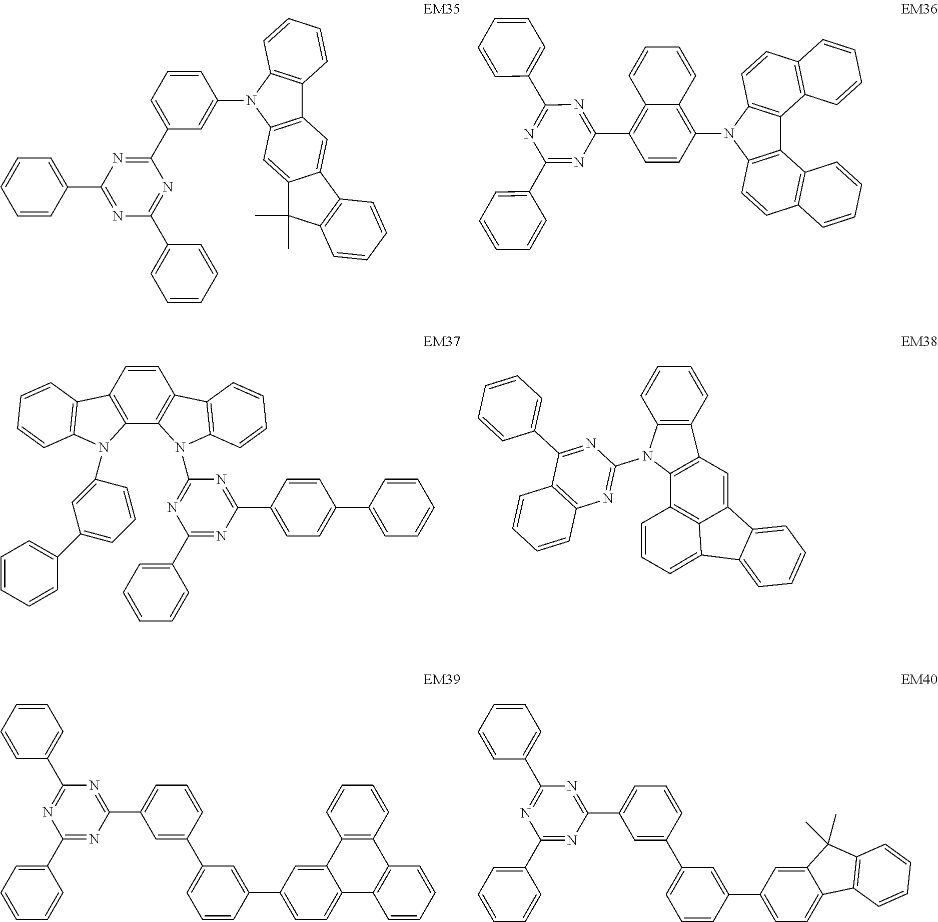

An electron transport material can be selected from materials that can transport an electron injected from a negative electrode to a light-emitting layer and is selected in consideration of the balance with the hole mobility of a hole transport material and the like. A material with electron transport ability may be an oxadiazole derivative, an oxazole derivative, a pyrazine derivative, a triazole derivative, a triazine derivative, a quinoline derivative, a quinoxaline derivative, a phenanthroline derivative, an organoaluminum complex, and a fused-ring compound (for example, a fluorene derivative, a naphthalene derivative, a chrysene derivative, an anthracene derivative, or the like). Furthermore, the electron transport material is also suitable for use in a hole-blocking layer. Specific examples of a compound that can be used as the electron transport material include, but are not limited to, the following.

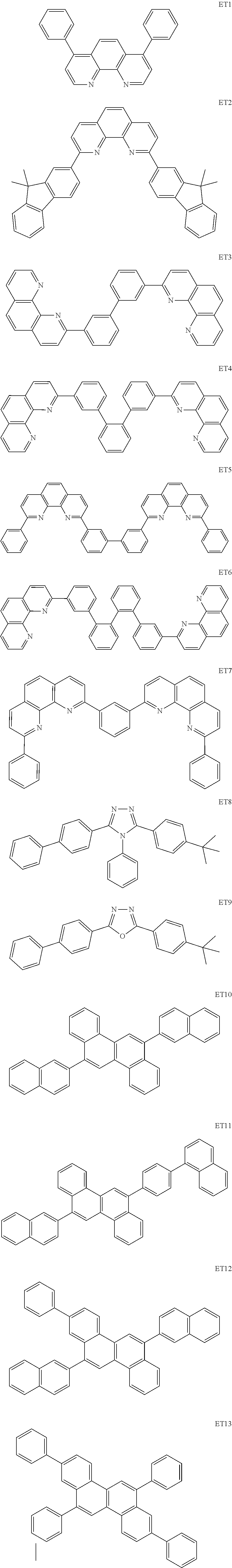

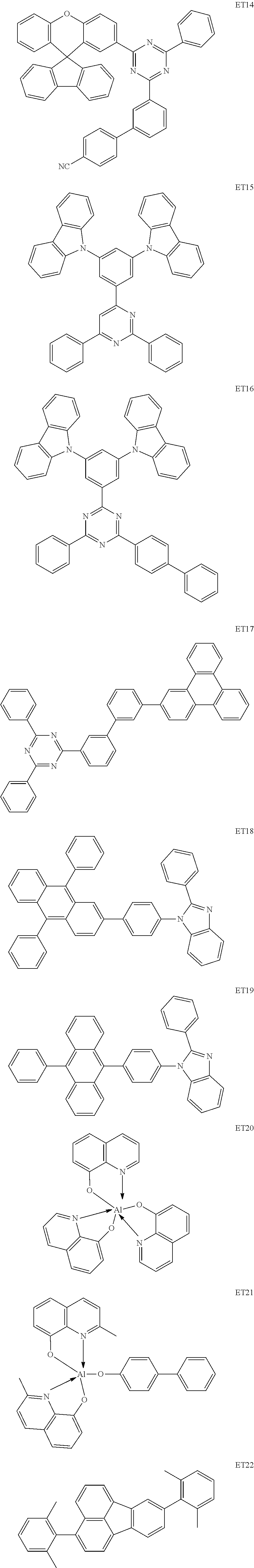

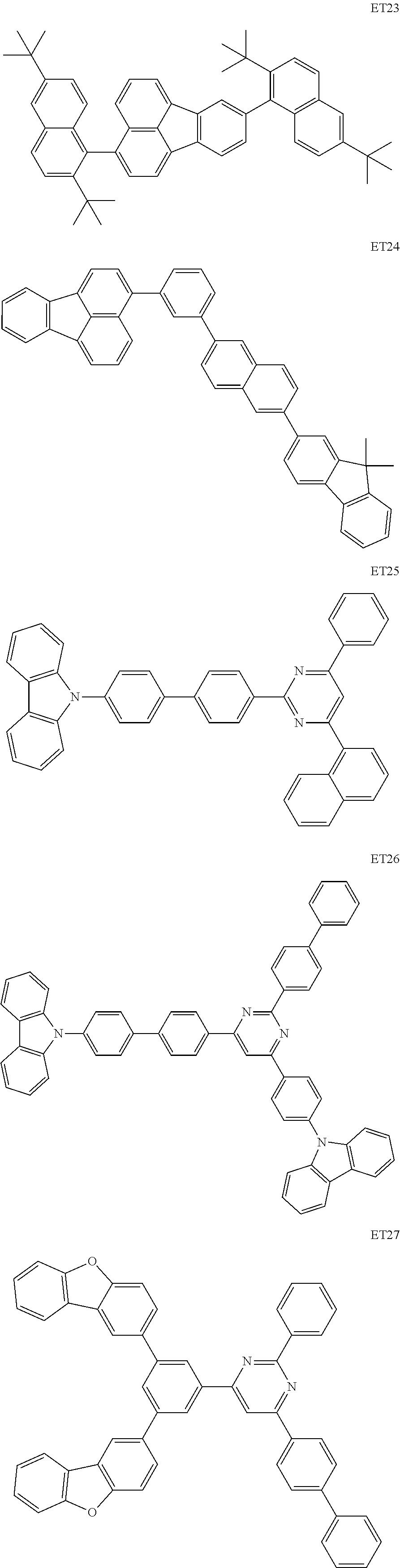

An electron injection material can be selected from materials that can easily inject an electron from a negative electrode and is selected in consideration of the balance with the hole injection properties and the like. An n-type dopant or a reducing dopant may be contained as an organic compound. Examples thereof include a compound containing an alkali metal, such as lithium fluoride, a lithium complex, such as lithium quinolinol, a benzimidazolidene derivative, an imidazolidene derivative, a fulvalene derivative, and an acridine derivative. It can also be used in combination with the electron transport material.

The organic compound layer (the hole injection layer, the hole transport layer, the electron-blocking layer, the light-emitting layer, the hole-blocking layer, the electron transport layer, the electron injection layer, and the like) of an organic light-emitting element according to the present disclosure is formed by the following methods.

For example, a dry process, such as a vacuum deposition method, an ionized deposition method, sputtering, or plasma, can be used. Instead of the dry process, a wet process may also be employed in which a layer is formed by a known coating method (for example, spin coating, dipping, a casting method, an LB method, an ink jet method, or the like) using an appropriate solvent.

A layer formed by a vacuum deposition method, a solution coating method, or the like undergoes little crystallization or the like and has high temporal stability. When a film is formed by a coating method, the film may also be formed in combination with an appropriate binder resin.

The binder resin may be, but is not limited to, a polyvinylcarbazole resin, a polycarbonate resin, a polyester resin, an ABS resin, an acrylic resin, a polyimide resin, a phenolic resin, an epoxy resin, a silicone resin, or a urea resin.

These binder resins may be used alone or in combination as a homopolymer or a copolymer. If necessary, an additive agent, such as a known plasticizer, oxidation inhibitor, and/or ultraviolet absorbent, may also be used.

<Constituent Materials Other than Organic Compound Layer>

An organic light-emitting element includes an insulating layer, a first electrode, an organic compound layer, and a second electrode on a substrate. A protective layer, a color filter, a microlens, or the like may be provided on the second electrode. When a color filter is provided, a planarization layer may be provided between the color filter and the protective layer. The planarization layer may be composed of an acrylic resin or the like. The same applies to the planarization layer provided between the color filter and the microlens.

### [Substrate]

The substrate may be formed of quartz, glass, a silicon wafer, resin, metal, or the like. The substrate may have a switching element, such as a transistor, and wiring, on which an insulating layer may be provided. The insulating layer may be composed of any material, provided that the insulating layer can have a contact hole for wiring between the insulating layer and the first electrode and is insulated from unconnected wires. For example, the insulating layer may be formed of a resin, such as polyimide, silicon oxide, silicon nitride, or the like.

### [Electrode]

A pair of electrodes can be used as electrodes. The pair of electrodes may be a positive electrode and a negative electrode. When an electric field is applied in a direction in which the organic light-emitting element emits light, an electrode with a high electric potential is a positive electrode, and the other electrode is a negative electrode. In other words, the electrode that supplies a hole to the light-emitting layer is a positive electrode, and the electrode that supplies an electron to the light-emitting layer is a negative electrode.

A constituent material of the positive electrode can have as large a work function as possible. Examples thereof include a metal element, such as gold, platinum, silver, copper, nickel, palladium, cobalt, selenium, vanadium, or tungsten, a mixture thereof, an alloy thereof, and a metal oxide, such as tin oxide, zinc oxide, indium oxide, indium tin oxide (ITO), or indium zinc oxide. An electrically conductive polymer, such as polyaniline, polypyrrole, or polythiophene, may also be used.

These electrode materials may be used alone or in combination. The positive electrode may be composed of a single layer or a plurality of layers.

When used as a reflective electrode, for example, chromium, aluminum, silver, titanium, tungsten, molybdenum, an alloy thereof, a laminate thereof, or the like can be used. These materials can also function as a reflective film that does not have a role as an electrode. When used as a transparent electrode, an oxide transparent electroconductive layer, such as indium tin oxide (ITO) or indium zinc oxide, can be used. However, the present disclosure is not limited thereto. The electrodes may be formed by photolithography.

On the other hand, a constituent material of the negative electrode can be a material with a small work function. For example, an alkali metal, such as lithium, an alkaline-earth metal, such as calcium, a metal element, such as aluminum, titanium, manganese, silver, lead, or chromium, or a mixture thereof may be used. An alloy of these metal elements may also be used. For example, magnesium-silver, aluminum-lithium, aluminum-magnesium, silver-copper, or zinc-silver may be used. A metal oxide, such as indium tin oxide (ITO), may also be used. These electrode materials may be used alone or in combination. The negative electrode may be composed of a single layer or a plurality of layers. In particular, silver can be used, and a silver alloy can be used to reduce the aggregation of silver. As long as the aggregation of silver can be reduced, the alloy may have any ratio. For example, the ratio of silver to another metal may be 1:1, 3:1, or the like.

The negative electrode may be, but is not limited to, an oxide electroconductive layer, such as ITO, for a top emission element or a reflective electrode, such as aluminum (Al), for a bottom emission element. The negative electrode may be formed by any method. A direct-current or alternating-current sputtering method can achieve good film coverage and easily decrease resistance.

### [Protective Layer]

A protective layer may be provided on the second electrode. For example, a glass sheet with a moisture absorbent may be attached to the second electrode to decrease the amount of water or the like entering the organic compound layer and to reduce the occurrence of display defects. In another embodiment, a passivation film of silicon nitride or the like may be provided on the second electrode to decrease the amount of water or the like entering the organic compound layer. For example, the second electrode may be formed and then transferred to another chamber without breaking the vacuum, and a silicon nitride film with a thickness of 2 μm may be formed as a protective layer by a chemical vapor deposition (CVD) method. The film formation by the CVD method may be followed by the formation of a protective layer by an atomic layer deposition (ALD) method. A film formed by the ALD method may be formed of any material, such as silicon nitride, silicon oxide, or aluminum oxide. Silicon nitride may be further formed by the CVD method on the film formed by the ALD method. The film formed by the ALD method may have a smaller thickness than the film formed by the CVD method. More specifically, the thickness may be 50% or less or even 10% or less.

### [Color Filter]

A color filter may be provided on the protective layer. For example, a color filter that matches the size of the organic light-emitting element may be provided on another substrate and may be bonded to the substrate on which the organic light-emitting element is provided, or a color filter may be patterned on the protective layer by photolithography. The color filter may be composed of a polymer.

### [Planarization Layer]

A planarization layer may be provided between the color filter and the protective layer. The planarization layer is provided to reduce the roughness of the underlayer. The planarization layer is sometimes referred to as a material resin layer with any purpose. The planarization layer may be composed of an organic compound, a low-molecular-weight compound, or a high-molecular-weight compound and can be composed of a high-molecular-weight compound.

The planarization layer may be provided above and below the color filter, and the constituent materials thereof may be the same or different. Specific examples include a polyvinylcarbazole resin, a polycarbonate resin, a polyester resin, an ABS resin, an acrylic resin, a polyimide resin, a phenolic resin, an epoxy resin, a silicone resin, and a urea resin.

### [Microlens]

An organic light-emitting element or an organic light-emitting apparatus with an organic light-emitting element may include an optical member, such as a microlens, on the light output side. The microlens may be composed of an acrylic resin, an epoxy resin, or the like. The microlens may be used to increase the amount of light extracted from the organic light-emitting element or the organic light-emitting apparatus and control the direction of the extracted light. The microlens may have a hemispherical shape. For a hemispherical microlens, the vertex of the microlens is a contact point between the hemisphere and a tangent line parallel to the insulating layer among the tangent lines in contact with the hemisphere. The vertex of the microlens in any cross-sectional view can be determined in the same manner. More specifically, the vertex of the microlens in a cross-sectional view is a contact point between the semicircle of the microlens and a tangent line parallel to the insulating layer among the tangent lines in contact with the semicircle.

The midpoint of the microlens can also be defined. In a cross section of the microlens, a midpoint of a line segment from one end point to the other end point of the arc can be referred to as a midpoint of the microlens. A cross section in which the vertex and the midpoint are determined may be perpendicular to the insulating layer.

### [Opposite Substrate]

An opposite substrate may be provided on the planarization layer. The opposite substrate is so called because it faces the substrate. The opposite substrate may be composed of the same material as the substrate. When the substrate is a first substrate, the opposite substrate may be a second substrate.

### [Pixel Circuit]

An organic light-emitting apparatus including an organic light-emitting element may include a pixel circuit coupled to the organic light-emitting element. The pixel circuit may be of an active matrix type, which independently controls the light emission of a plurality of light-emitting elements. The active-matrix circuit may be voltage programmed or current programmed. The drive circuit has a pixel circuit for each pixel. The pixel circuit may include a light-emitting element, a transistor for controlling the luminous brightness of the light-emitting element, a transistor for controlling light emission timing, a capacitor for holding the gate voltage of the transistor for controlling the luminous brightness, and a transistor for GND connection without through the light-emitting element.

A light-emitting apparatus includes a display region and a peripheral region around the display region. The display region includes the pixel circuit, and the peripheral region includes a display control circuit. The mobility of a transistor constituting the pixel circuit may be smaller than the mobility of a transistor constituting the display control circuit. The gradient of the current-voltage characteristics of a transistor constituting the pixel circuit may be smaller than the gradient of the current-voltage characteristics of a transistor constituting the display control circuit. The gradient of the current-voltage characteristics can be determined by so-called Vg-Ig characteristics. A transistor constituting the pixel circuit is a transistor coupled to a light-emitting element.

### [Pixel]

An organic light-emitting apparatus including an organic light-emitting element may have a plurality of pixels. Each pixel has subpixels that emit light of different colors. For example, the subpixels may have RGB emission colors.

In each pixel, a region also referred to as a pixel aperture emits light. This region is the same as the first region. The pixel aperture may be 15.0 μm or less and 5.0 μm or more. More specifically, the pixel aperture may be 11.0 μm, 9.5 μm, 7.4 μm, 6.4 μm, or the like. The distance between the subpixels may be 10.0 μm or less, more specifically, 8 μm, 7.4 μm, or 6.4 μm.

The pixels may be arranged in a known form in a plan view. Examples include a stripe arrangement, a delta arrangement, a PenTile arrangement, and a Bayer arrangement. Each subpixel may have any known shape in a plan view. Examples include quadrangles, such as a rectangle and a rhombus, and a hexagon. As a matter of course, the rectangle also includes a figure that is not strictly rectangular but is close to rectangular. The shape of each subpixel and the pixel array can be used in combination.

### <Applications of Organic Light-Emitting Element>

An organic light-emitting element according to the present disclosure can be used as a constituent of a display apparatus or a lighting apparatus. Other applications include an exposure light source for an electrophotographic image-forming apparatus, a backlight for a liquid crystal display, and a light-emitting apparatus with a color filter in a white light source.

The display apparatus may be an image-information-processing apparatus that includes an image input unit for inputting image information from an area CCD, a linear CCD, a memory card, or the like, includes an information processing unit for processing the input information, and displays an input image on a display unit. The display apparatus may have a plurality of pixels, and at least one of the pixels may include an organic light-emitting element according to the present disclosure and a transistor coupled to the organic light-emitting element.

A display unit of an imaging apparatus or an ink jet printer may have a touch panel function. A driving system of the touch panel function may be, but is not limited to, an infrared radiation system, an electrostatic capacitance system, a resistive film system, or an electromagnetic induction system. The display apparatus may be used for a display unit of a multifunction printer.

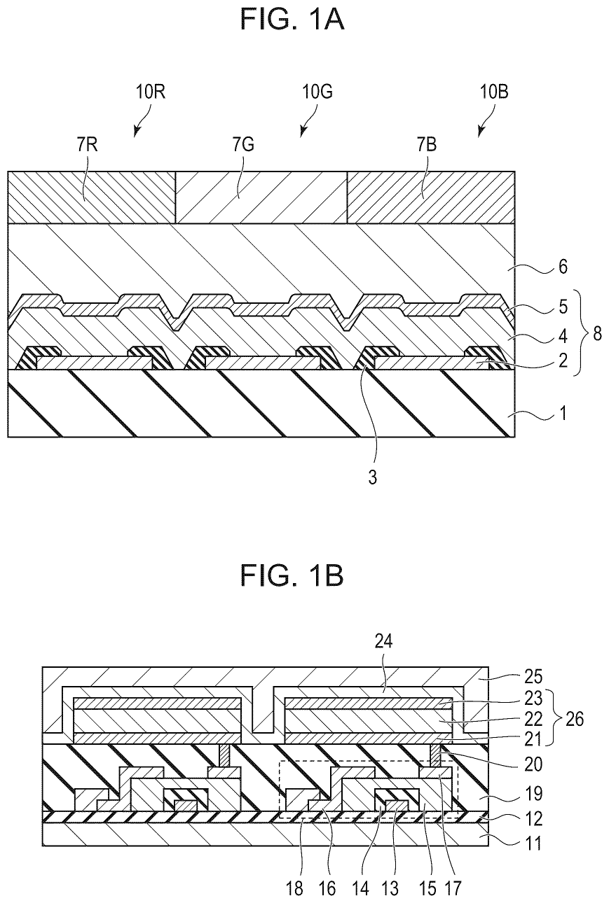

Next, a display apparatus according to the present disclosure is described below with reference to the accompanying drawings. FIGS. 1A and 1B are schematic cross-sectional views of an example of a display apparatus that includes an organic light-emitting element and a transistor coupled to the organic light-emitting element. The transistor is an example of an active element. The transistor may be a thin-film transistor (TFT).

FIG. 1A illustrates an example of a pixel serving as a constructional element of a display apparatus according to the present disclosure. The pixel has subpixels 10. The subpixels are 10R, 10G, and 10B with different emission colors. The emission colors may be distinguished by the wavelength of light emitted from the light-emitting layer, or light emitted from each subpixel may be selectively transmitted or color-converted with a color filter or the like. Each of the subpixels 10 includes a reflective electrode serving as a first electrode 2, an insulating layer 3 covering an end of the first electrode 2, an organic compound layer 4 covering the first electrode 2 and the insulating layer 3, a transparent electrode serving as a second electrode 5, a protective layer 6, and a color filter 7 on an interlayer insulating layer 1.

A transistor and/or a capacitor element may be provided under or inside the interlayer insulating layer 1. The transistor and the first electrode 2 may be electrically connected via a contact hole (not shown) or the like.

The insulating layer 3 is also referred to as a bank or a pixel separation film. The insulating layer 3 covers the ends of the first electrode 2 and surrounds the first electrode 2. A portion not covered with the insulating layer 3 is in contact with the organic compound layer 4 and serves as a light-emitting region.

The second electrode 5 may be a transparent electrode, a reflective electrode, or a semitransparent electrode.

The protective layer 6 reduces the penetration of moisture into the organic compound layer 4. The protective layer 6 is illustrated as a single layer but may be a plurality of layers. The protective layer 6 may include an inorganic compound layer and an organic compound layer.

The color filter 7 is divided into 7R, 7G, and 7B according to the color. The color filter 7 may be formed on a planarizing film (not shown). Furthermore, a resin protective layer (not shown) may be provided on the color filter 7. The color filter 7 may be formed on the protective layer 6. Alternatively, the color filter 7 may be bonded after being provided on an opposite substrate, such as a glass substrate.

A display apparatus in FIG. 1B includes an organic light-emitting element 26 and a TFT 18 as an example of a transistor. The display apparatus includes a substrate 11 made of glass, silicon, or the like and an insulating layer 12 on the substrate 11. An active element, such as the TFT 18, and a gate electrode 13, a gate-insulating film 14, and a semiconductor layer 15 of the active element are disposed on the insulating layer 12. The TFT 18 is also composed of a drain electrode 16 and a source electrode 17. The TFT 18 is covered with an insulating film 19. A positive electrode 21 of the organic light-emitting element 26 is coupled to the source electrode 17 through a contact hole 20 formed in the insulating film 19.

Electrical connection between the electrodes of the organic light-emitting element 26 (the positive electrode 21 and a negative electrode 23) and the electrodes of the TFT 18 (the source electrode 17 and the drain electrode 16) is not limited to that illustrated in FIG. 1B. More specifically, it is only necessary to electrically connect either the positive electrode 21 or the negative electrode 23 to either the source electrode 17 or the drain electrode 16 of the TFT 18. TFT refers to a thin-film transistor.

Although an organic compound layer 22 is a single layer in the display apparatus illustrated in FIG. 1B, the organic compound layer 22 may be composed of a plurality of layers. The negative electrode 23 is covered with a first protective layer 24 and a second protective layer 25 for reducing degradation of the organic light-emitting element 26.

Although a transistor is used as a switching element in the display apparatus in FIG. 1B, another switching element may be used instead.

The transistor used in the display apparatus in FIG. 1B is not limited to a transistor including a single crystal silicon wafer and may also be a thin-film transistor including an active layer on an insulating surface of a substrate. The active layer may be single-crystal silicon, non-single-crystal silicon, such as amorphous silicon or microcrystalline silicon, or a non-single-crystal oxide semiconductor, such as indium zinc oxide or indium gallium zinc oxide. The thin-film transistor is also referred to as a TFT element.

The transistor in the display apparatus in FIG. 1B may be formed within a substrate, such as a Si substrate. The phrase “formed within a substrate” means that the substrate, such as a Si substrate, itself is processed to form the transistor. Thus, the transistor within the substrate can be considered that the substrate and the transistor are integrally formed.

In the organic light-emitting element according to the present embodiment, the luminous brightness is controlled with the TFT, which is an example of a switching element. The organic light-emitting element can be provided in a plurality of planes to display an image at each luminous brightness. The switching element according to the present embodiment is not limited to the TFT and may be a transistor formed of low-temperature polysilicon or an active-matrix driver formed on a substrate, such as a Si substrate. The phrase “on a substrate” may also be referred to as “within a substrate”. Whether a transistor is provided within a substrate or a TFT is used depends on the size of a display unit. For example, for an approximately 0.5-inch display unit, an organic light-emitting element can be provided on a Si substrate.

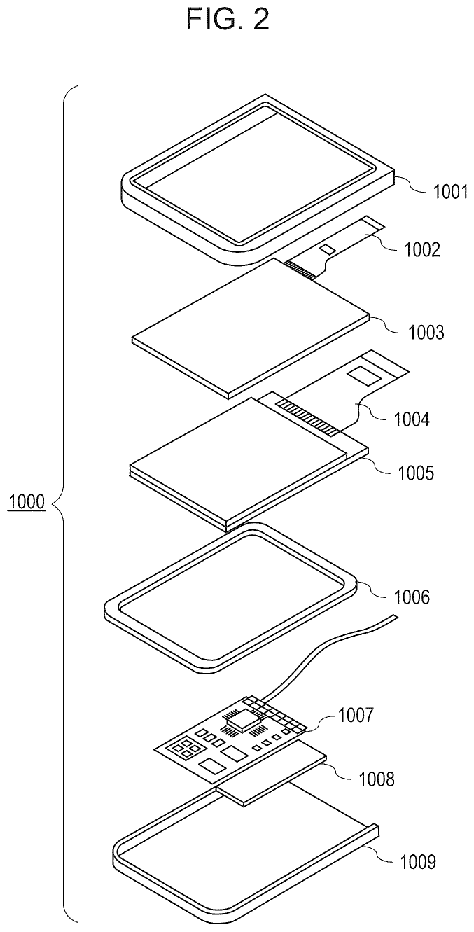

FIG. 2 is a schematic view of an example of a display apparatus according to the present embodiment. A display apparatus 1000 may include a touch panel 1003, a display panel 1005, a frame 1006, a circuit substrate 1007, and a battery 1008 between an upper cover 1001 and a lower cover 1009. The touch panel 1003 and the display panel 1005 are coupled to flexible print circuits FPC 1002 and 1004, respectively. Transistors are printed on the circuit substrate 1007. The battery 1008 is not necessarily provided when the display apparatus is not a mobile device, or may be provided at another position even when the display apparatus is a mobile device.

The display apparatus according to the present embodiment may include color filters of red, green, and blue colors. In the color filters, the red, green, and blue colors may be arranged in a delta arrangement.

The display apparatus according to the present embodiment may be used for a display unit of a mobile terminal. Such a display apparatus may have both a display function and an operation function. The mobile terminal may be a mobile phone, such as a smartphone, a tablet, a head-mounted display, or the like.

The display apparatus according to the present embodiment may be used for a display unit of an imaging apparatus that includes an optical unit with a plurality of lenses and an imaging element for receiving light passing through the optical unit. The imaging apparatus may include a display unit for displaying information acquired by the imaging element. The display unit may be a display unit exposed outside from the imaging apparatus or a display unit located in a finder. The imaging apparatus may be a digital camera or a digital camcorder.

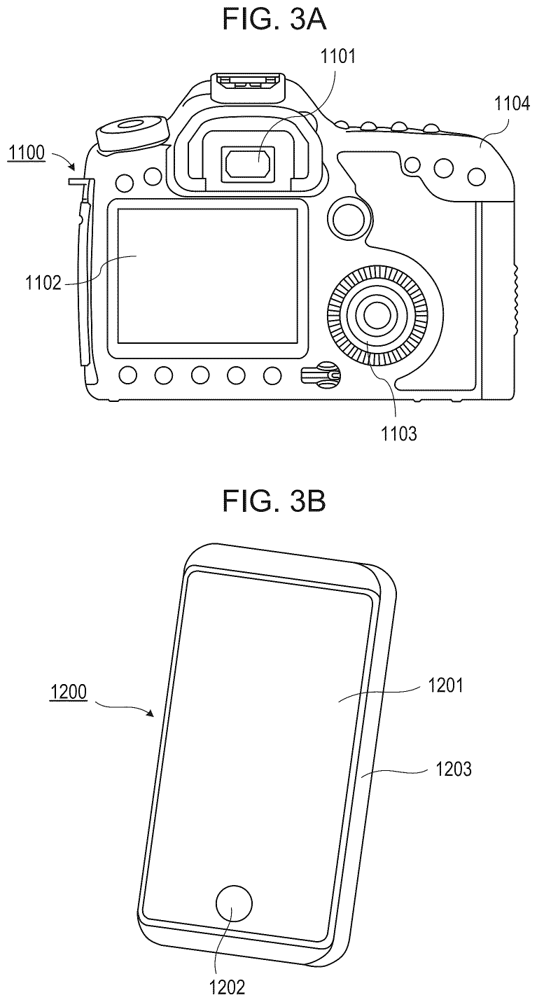

FIG. 3A is a schematic view of an example of an imaging apparatus according to the present embodiment. An imaging apparatus 1100 may include a viewfinder 1101, a rear display 1102, an operating unit 1103, and a housing 1104. The viewfinder 1101 includes the display apparatus according to the present embodiment. In this case, the display apparatus may display environmental information, imaging instructions, and the like as well as an image to be captured. The environmental information may include the intensity of external light, the direction of external light, the travel speed of the photographic subject, the possibility that the photographic subject is shielded by a shielding material, and the like.

Because the appropriate timing for imaging is a short time, it is better to display information as early as possible. Thus, a display apparatus including an organic light-emitting element according to the present disclosure can be used. This is because the organic light-emitting element has a high response speed. A display apparatus including the organic light-emitting element can be more suitably used than these apparatuses and liquid crystal displays that require a high display speed.

The imaging apparatus 1100 includes an optical unit (not shown). The optical unit has a plurality of lenses and focuses an image on an imaging element in the housing 1104. The focus of the lenses can be adjusted by adjusting their relative positions. This operation can also be automatically performed. The imaging apparatus may also be referred to as a photoelectric conversion apparatus. The photoelectric conversion apparatus can have, as an imaging method, a method of detecting a difference from a previous image, a method of cutting out a permanently recorded image, or the like, instead of taking an image one after another.

FIG. 3B is a schematic view of an example of electronic equipment according to the present embodiment. Electronic equipment 1200 includes a display unit 1201, an operating unit 1202, and a housing 1203. The housing 1203 may include a circuit, a printed circuit board including the circuit, a battery, and a communication unit. The operating unit 1202 may be a button or a touch panel response unit. The operating unit 1202 may be a biometric recognition unit that recognizes a fingerprint and releases the lock. Electronic equipment with a communication unit may also be referred to as communication equipment. The electronic equipment 1200 may have a lens and an imaging element and thereby further have a camera function. An image captured by the camera function is displayed on the display unit 1201. The electronic equipment 1200 may be a smartphone, a notebook computer, or the like.

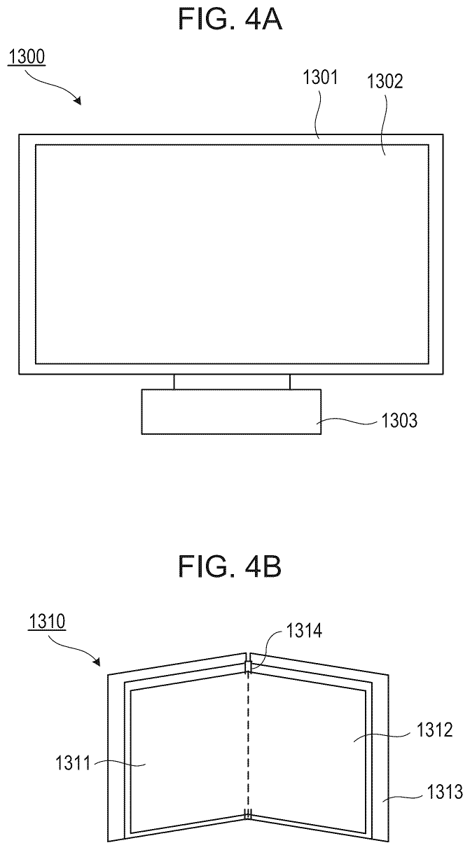

FIGS. 4A and 4B are schematic views of an example of the display apparatus according to the present embodiment. FIG. 4A illustrates a display apparatus, such as a television monitor or a PC monitor. A display apparatus 1300 includes a frame 1301 and a display unit 1302. An organic light-emitting element according to the present disclosure is used for the display unit 1302. Furthermore, a base 1303 that supports the frame 1301 and the display unit 1302 is provided. The base 1303 is not limited to the structure illustrated in FIG. 4A. The lower side of the frame 1301 may also serve as the base. The frame 1301 and the display unit 1302 may be bent. The radius of curvature thereof may be 5000 mm or more and 6000 mm or less.

FIG. 4B is a schematic view of another example of the display apparatus according to the present embodiment. A display apparatus 1310 in FIG. 4B is configured to be foldable and is a so-called foldable display apparatus. The display apparatus 1310 includes a first display unit 1311, a second display unit 1312, a housing 1313, and a folding point 1314. The first display unit 1311 and the second display unit 1312 have an organic light-emitting element according to the present disclosure. The first display unit 1311 and the second display unit 1312 may be a single display apparatus without a joint. The first display unit 1311 and the second display unit 1312 can be divided by the folding point. The first display unit 1311 and the second display unit 1312 may display different images or one image.

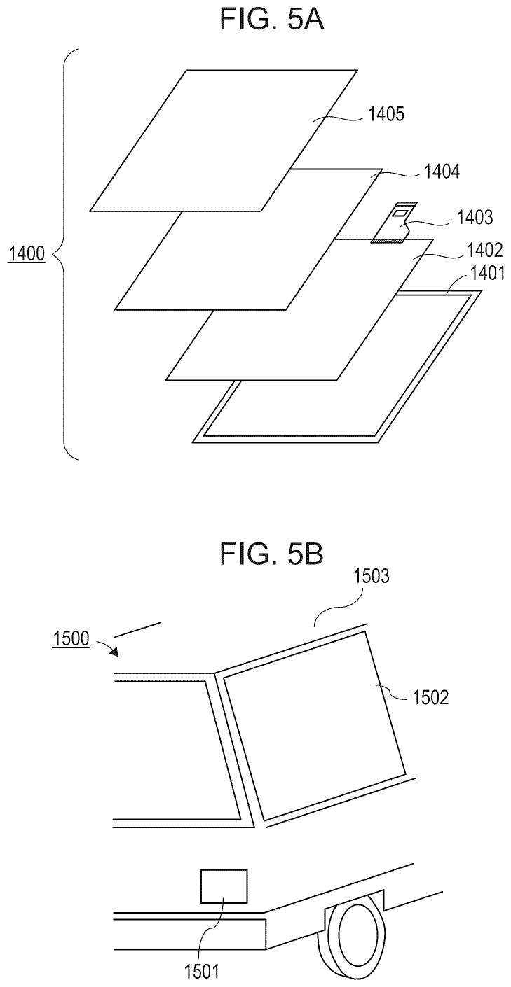

FIG. 5A is a schematic view of an example of a lighting apparatus according to the present embodiment. A lighting apparatus 1400 may include a housing 1401, a light source 1402, a circuit substrate 1403, an optical filter 1404 that transmits light emitted by the light source 1402, and a light-diffusing unit 1405. The light source 1402 has an organic light-emitting element according to the present disclosure. The optical filter 1404 may be a filter that improves the color rendering properties of the light source. The light-diffusing unit 1405 can effectively diffuse light from the light source and widely spread light as in illumination. The optical filter 1404 and the light-diffusing unit 1405 may be provided on the light output side of the lighting apparatus. If necessary, a cover may be provided on the outermost side.

For example, the lighting apparatus is an interior lighting apparatus. The lighting apparatus may emit white light, neutral white light, or light of any color from blue to red. The lighting apparatus may have a light control circuit for controlling such light. The lighting apparatus includes an organic light-emitting element according to the present disclosure and a power supply circuit coupled thereto. The power supply circuit is a circuit that converts an AC voltage to a DC voltage. White has a color temperature of 4200 K, and neutral white has a color temperature of 5000 K. The lighting apparatus may have a color filter.

The lighting apparatus according to the present embodiment may include a heat dissipation unit. The heat dissipation unit releases heat from the apparatus to the outside and may be a metal or liquid silicon with a high specific heat.

FIG. 5B is a schematic view of an automobile as an example of a moving body according to the present embodiment. The automobile has a taillight as an example of a lamp. An automobile 1500 may have a taillight 1501, which comes on when a brake operation or the like is performed.

The taillight 1501 has an organic light-emitting element according to the present disclosure. The taillight 1501 may include a protective member for protecting the organic light-emitting element. The protective member may be formed of any transparent material with moderately high strength and can be formed of polycarbonate or the like. The polycarbonate may be mixed with a furan dicarboxylic acid derivative, an acrylonitrile derivative, or the like.

The automobile 1500 may have a body 1503 and a window 1502 on the body 1503. The window 1502 may be a transparent display as long as it is not a window for checking the front and rear of the automobile. The transparent display has an organic light-emitting element according to the present disclosure. In such a case, constituent materials, such as electrodes, of the organic light-emitting element are transparent materials.

The moving body according to the present embodiment may be a ship, an aircraft, a drone, or the like. The moving body may include a body and a lamp provided on the body, and the lamp may emit light for notifying the position of the body. The lamp has an organic light-emitting element according to the present disclosure.

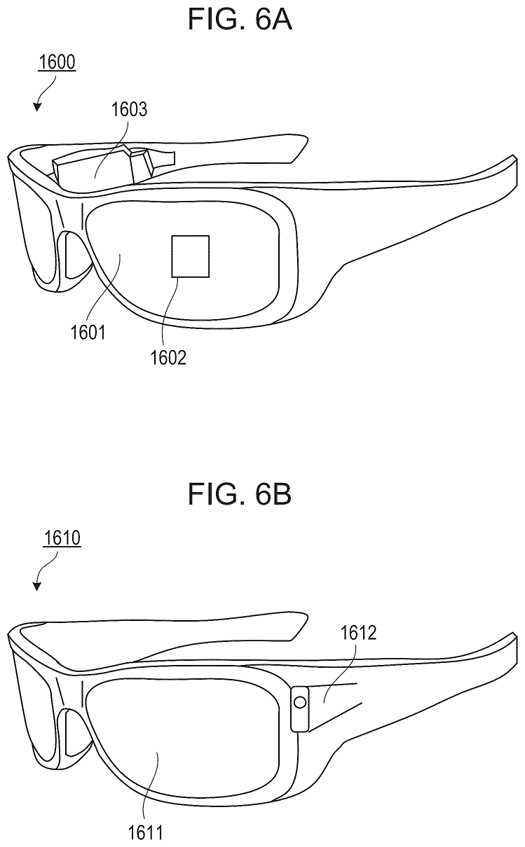

Application examples of the display apparatus according to one of the embodiments are described below with reference to FIGS. 6A and 6B. The display apparatus can be applied to a system that can be worn as a wearable device, such as smart glasses, a head-mounted display (HMD), or smart contact lenses. An imaging and displaying apparatus used in such an application example includes an imaging apparatus that can photoelectrically convert visible light and a display apparatus that can emit visible light.

FIG. 6A is a schematic view of an example of a wearable device according to an embodiment of the present disclosure. Glasses 1600 (smart glasses) according to one application example will be described below with reference to FIG. 6A. An imaging apparatus 1602, such as a complementary metal-oxide semiconductor (CMOS) sensor or a single-photon avalanche photodiode (SPAD), is provided on the front side of a lens 1601 of the glasses 1600. The display apparatus according to one of the embodiments is provided on the back side of the lens 1601.

The glasses 1600 further include a controller 1603. The controller 1603 functions as a power supply for supplying power to the imaging apparatus 1602 and the display apparatus. The controller 1603 controls the operation of the imaging apparatus 1602 and the display apparatus. The lens 1601 has an optical system for focusing light on the imaging apparatus 1602.

FIG. 6B is a schematic view of another example of a wearable device according to an embodiment of the present disclosure. Glasses 1610 (smart glasses) according to one application example will be described below with reference to FIG. 6B. The glasses 1610 have a controller 1612, which includes an imaging apparatus corresponding to the imaging apparatus 1602 of FIG. 6A and a display apparatus. A lens 1611 includes an optical system for projecting light from the imaging apparatus of the controller 1612 and the display apparatus, and an image is projected on the lens 1611. The controller 1612 functions as a power supply for supplying power to the imaging apparatus and the display apparatus and controls the operation of the imaging apparatus and the display apparatus.

The controller 1612 may include a line-of-sight detection unit for detecting the line of sight of the wearer. Infrared radiation may be used to detect the line of sight. An infrared radiation unit emits infrared light to an eyeball of a user who is gazing at a display image. Reflected infrared light from the eyeball is detected by an imaging unit including a light-receiving element to capture an image of the eyeball. A reduction unit for reducing light from the infrared radiation unit to a display unit in a plan view is provided to reduce degradation in image quality. The line of sight of the user for the display image is detected from the image of the eyeball captured by infrared imaging. Any known technique can be applied to line-of-sight detection using the captured image of the eyeball. For example, it is possible to use a line-of-sight detection method based on a Purkinje image obtained by the reflection of irradiation light by the cornea. More specifically, a line-of-sight detection process based on a pupil-corneal reflection method is performed. The line of sight of the user is detected by calculating a line-of-sight vector representing the direction (rotation angle) of an eyeball on the basis of an image of a pupil and a Purkinje image included in a captured image of the eyeball using the pupil-corneal reflection method.

A display apparatus according to an embodiment of the present disclosure may include an imaging apparatus including a light-receiving element and may control a display image on the basis of line-of-sight information of a user from the imaging apparatus. More specifically, on the basis of the line-of-sight information, the display apparatus determines a first visibility region at which the user gazes and a second visibility region other than the first visibility region. The first visibility region and the second visibility region may be determined by the controller of the display apparatus or may be received from an external controller. In the display region of the display apparatus, the first visibility region may be controlled to have higher display resolution than the second visibility region. In other words, the second visibility region may have lower resolution than the first visibility region.

The display region has a first display region and a second display region different from the first display region, and the priority of the first display region and the second display region depends on the line-of-sight information. The first visibility region and the second visibility region may be determined by the controller of the display apparatus or may be received from an external controller. A region with a higher priority may be controlled to have higher resolution than another region. In other words, a region with a lower priority may have lower resolution.

The first visibility region or a region with a higher priority may be determined by artificial intelligence (AI). The AI may be a model configured to estimate the angle of the line of sight and the distance to a target ahead of the line of sight from an image of an eyeball using the image of the eyeball and the direction in which the eyeball actually viewed in the image as teaching data. The AI program may be stored in the display apparatus, the imaging apparatus, or an external device. The AI program stored in an external device is transmitted to the display apparatus via communication.

For display control based on visual recognition detection, the present disclosure can be applied to smart glasses further having an imaging apparatus for imaging the outside. Smart glasses can display captured external information in real time.

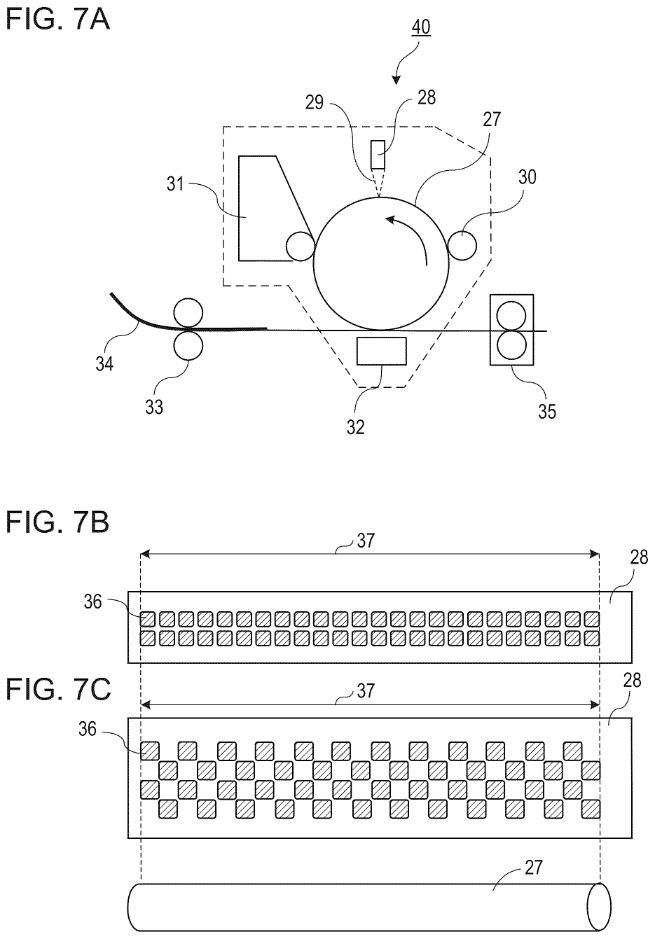

FIG. 7A is a schematic view of an example of an image-forming apparatus according to an embodiment of the present disclosure. An image-forming apparatus 40 is an electrophotographic image-forming apparatus and includes a photosensitive member 27, an exposure light source 28, a charging unit 30, a developing unit 31, a transfer unit 32, a conveying roller 33, and a fixing unit 35. The exposure light source 28 emits light 29, and an electrostatic latent image is formed on the surface of the photosensitive member 27. The exposure light source 28 includes an organic light-emitting element according to the present disclosure. The developing unit 31 contains toner and the like. The charging unit 30 electrifies the photosensitive member 27. The transfer unit 32 transfers a developed image onto a recording medium 34. The conveying roller 33 conveys the recording medium 34. The recording medium 34 is paper, for example. The fixing unit 35 fixes an image on the recording medium 34.

FIGS. 7A and 7B are schematic views of the exposure light source 28, wherein a plurality of light-emitting portions 36 are arranged on a long substrate. An arrow 37 indicates a direction parallel to the axis of the photosensitive member and indicates a column direction in which the organic light-emitting elements are arranged. This column direction is the same as the direction of the rotation axis of the photosensitive member 27. This direction can also be referred to as the major-axis direction of the photosensitive member 27. In FIG. 7B, the light-emitting portions 36 are arranged in the major-axis direction of the photosensitive member 27. In FIG. 7C, unlike FIG. 7B, the light-emitting portions 36 are alternately arranged in the column direction in the first and second columns. The first and second columns are arranged at different positions in the row direction. In the first column, the light-emitting portions 36 are arranged at intervals. In the second column, the light-emitting portions 36 are arranged at positions corresponding to the spaces between the light-emitting portions 36 of the first column. Thus, the light-emitting portions 36 are also arranged at intervals in the row direction. The arrangement in FIG. 7C can also be referred to as a grid-like pattern, a staggered pattern, or a checkered pattern, for example.

As described above, an apparatus including the organic light-emitting element according to the present embodiment can be used to stably display a high-quality image for extended periods.

### EXEMPLARY EMBODIMENTS

The present disclosure is described below with exemplary embodiments. However, the present disclosure is not limited to these exemplary embodiments.

### Exemplary Embodiment 1

Exemplary Compound A1 was synthesized in accordance with the following reaction formula.

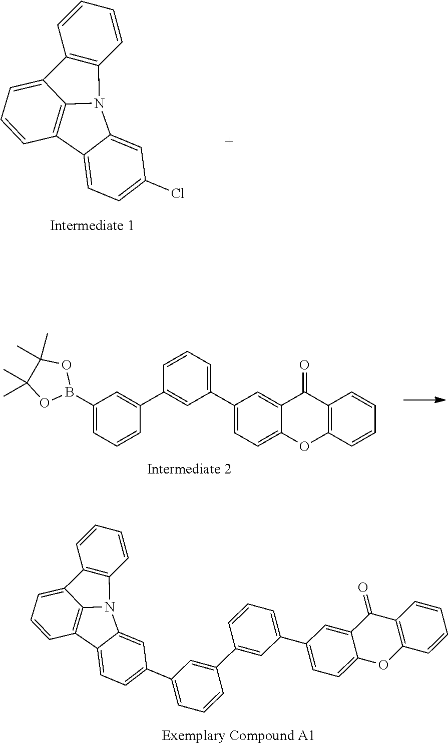 [SMILES: Clc1ccc2c3cccc4c5ccccc5n(c2c1)c43 ; CC1(C)OB(c2cccc(-c3cccc(-c4ccc5oc6ccccc6c(=O)c5c4)c3)c2)OC1(C)C ; O=c1c2ccccc2oc2ccc(-c3cccc(-c4cccc(-c5ccc6c7cccc8c9ccccc9n(c6c5)c87)c4)c3)cc12]

A 100-ml recovery flask was charged with the following reagent(s) and solvent(s).
Intermediate 1:1.00 g (3.63 mmol)
Intermediate 2:1.72 g (3.63 mmol)
Palladium (II) acetate (Pd(OAc)₂): 25 mg (0.11 mmol)
2-dicyclohexylphosphino-2′,6′-dimethoxybiphenyl(s-phos): 163 mg (0.36 mmol)
K₃PO₄: 1.54 g (7.26 mmol)
Toluene: 30 ml
Water: 1 ml

Next, the reaction solution was heated to 90° C. in a nitrogen stream and was stirred at this temperature (90° C.) for 5 hours. After completion of the reaction, methanol was added thereto, and the product was filtered to prepare a crude product as a residue. The residue was purified by silica gel column chromatography (chlorobenzene) and was then recrystallized using xylene to prepare 1.49 mg of Exemplary Compound A1 (yield: 70%).

Exemplary Compound A1 was subjected to mass spectrometry with MALDI-TOF-MS (Autoflex LRF manufactured by Bruker). The measured value was m/z=587, and the calculated value was C₄₃H₂₅NO₂=587.

### Exemplary Embodiments 2 to 30

The exemplary compounds were synthesized in the same manner as in Exemplary Embodiment 1 except that the intermediate 1 of Exemplary Embodiment 1 was changed to a raw material 1 and the intermediate 2 was changed to a raw material 2. The raw material 1 and the raw material 2 of each exemplary embodiment are shown in Table 4.

The measured values m/z measured by mass spectrometry in the same manner as in Exemplary Embodiment 1 are also shown.

TABLE 4

| | Exem- | | | |
|---|---|---|---|---|
| Exemplary | plary | | | |
| embodi- | com- | | | |
| ment | pound | Raw material 1 | Raw material 2 | m/z |
| Exemplary embodi- ment 2 | A5 | 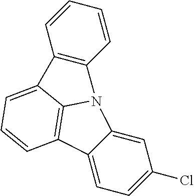 [SMILES: Clc1ccc2c3cccc4c5ccccc5n(c2c1)c43] | 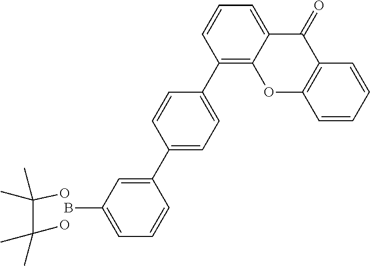 | 687 |
| Exemplary embodi- ment 3 | A8 | 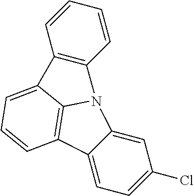 [SMILES: Clc1ccc2c3cccc4c5ccccc5n(c2c1)c43] | 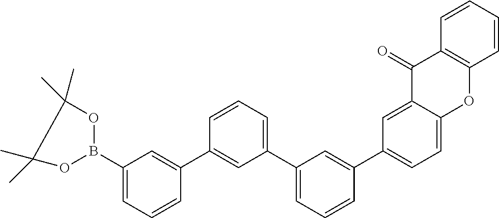 [SMILES: CC1(C)OB(c2cccc(-c3cccc(-c4cccc(-c5ccc6oc7ccccc7c(=O)c6c5)c4)c3)c2)OC1(C)C] | 663 |
| Exemplary embodi- ment 4 | A11 | 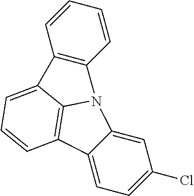 [SMILES: Clc1ccc2c3cccc4c5ccccc5n(c2c1)c43] | 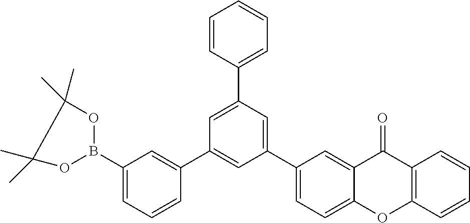 [SMILES: CC1(C)OB(c2cccc(-c3cc(-c4ccccc4)cc(-c4ccc5oc6ccccc6c(=O)c5c4)c3)c2)OC1(C)C] | 663 |
| Exemplary embodi- ment 5 | A12 | 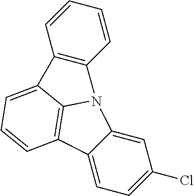 [SMILES: Clc1ccc2c3cccc4c5ccccc5n(c2c1)c43] | 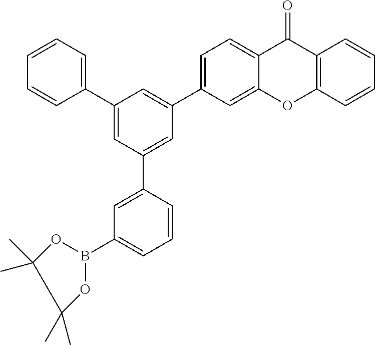 [SMILES: CC1(C)OB(c2cccc(-c3cc(-c4ccccc4)cc(-c4ccc5c(=O)c6ccccc6oc5c4)c3)c2)OC1(C)C] | 663 |
| Exemplary embodi- ment 6 | A13 | 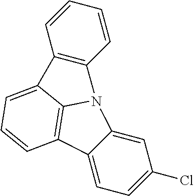 [SMILES: Clc1ccc2c3cccc4c5ccccc5n(c2c1)c43] | 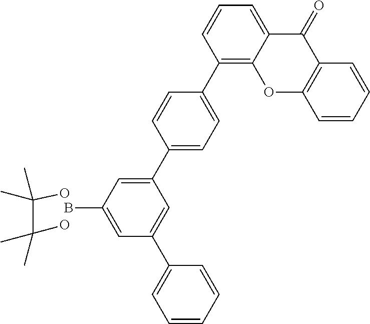 | 663 |
| Exemplary embodi- ment 7 | A15 | 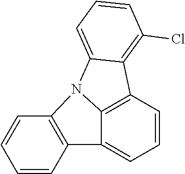 [SMILES: Clc1cccc2c1c1cccc3c4ccccc4n2c31] | 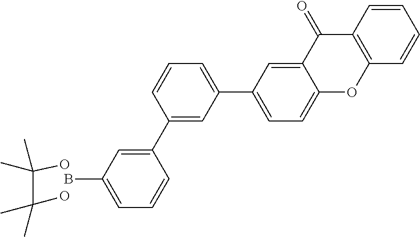 | 587 |
| Exemplary embodi- ment 8 | A21 | 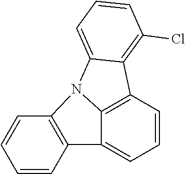 [SMILES: Clc1cccc2c1c1cccc3c4ccccc4n2c31] | 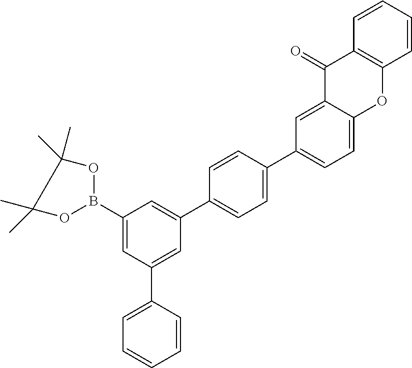 [SMILES: CC1(C)OB(c2cc(-c3ccccc3)cc(-c3ccc(-c4ccc5oc6ccccc6c(=O)c5c4)cc3)c2)OC1(C)C] | 663 |
| Exemplary embodi- ment 9 | A23 | 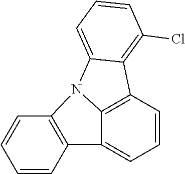 [SMILES: Clc1cccc2c1c1cccc3c4ccccc4n2c31] | 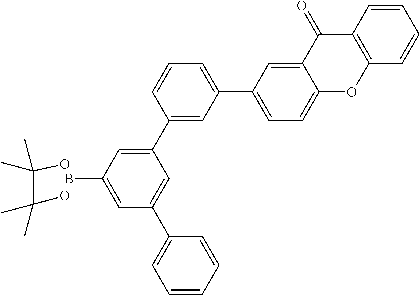 | 663 |
| Exemplary embodi- ment 10 | A27 | 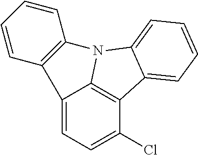 [SMILES: Clc1ccc2c3ccccc3n3c4ccccc4c1c23] | 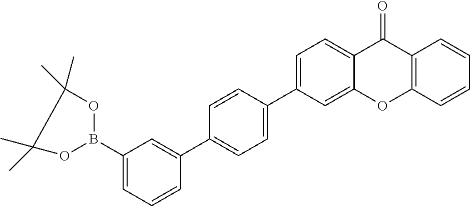 [SMILES: CC1(C)OB(c2cccc(-c3ccc(-c4ccc5c(=O)c6ccccc6oc5c4)cc3)c2)OC1(C)C] | 587 |

TABLE 5

| Exem- | Exem- | | | |
|---|---|---|---|---|
| plary | plary | | | |
| embodi- | com- | | | |
| ment | pound | Raw material 1 | Raw material 2 | m/z |
| Exem- plary embodi- ment 11 | A29 | 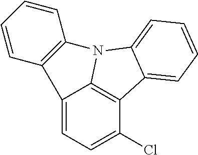 [SMILES: Clc1ccc2c3ccccc3n3c4ccccc4c1c23] | 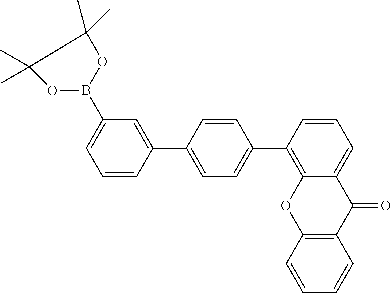 [SMILES: CC1(C)OB(c2cccc(-c3ccc(-c4cccc5c(=O)c6ccccc6oc45)cc3)c2)OC1(C)C] | 587 |
| Exem- plary embodi- ment 12 | A31 | 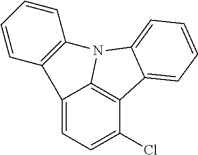 [SMILES: Clc1ccc2c3ccccc3n3c4ccccc4c1c23] | 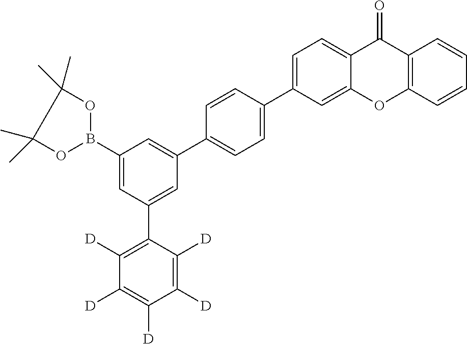 [SMILES: [2H]c1c([2H])c([2H])c(-c2cc(B3OC(C)(C)C(C)(C)O3)cc(-c3ccc(-c4ccc5c(=O)c6ccccc6oc5c4)cc3)c2)c([2H])c1[2H]] | 668 |
| Exem- plary embodi- ment 13 | B4 | 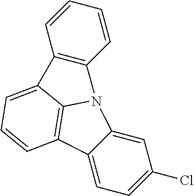 [SMILES: Clc1ccc2c3cccc4c5ccccc5n(c2c1)c43] | 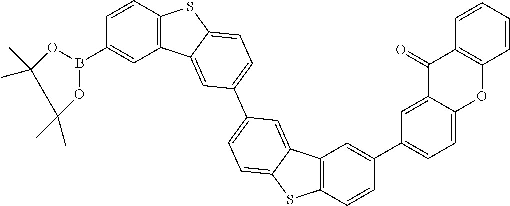 [SMILES: CC1(C)OB(c2ccc3sc4ccc(-c5ccc6sc7ccc(-c8ccc9oc%10ccccc%10c(=O)c9c8)cc7c6c5)cc4c3c2)OC1(C)C] | 799 |
| Exem- plary embodi- ment 14 | B8 | 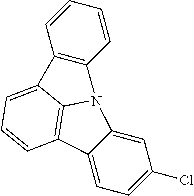 [SMILES: Clc1ccc2c3cccc4c5ccccc5n(c2c1)c43] | 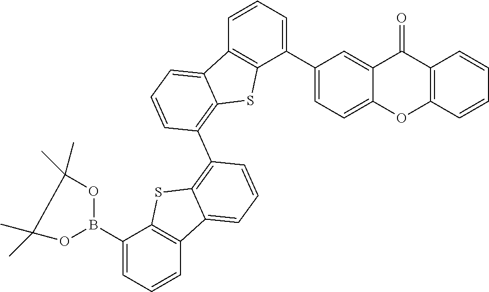 [SMILES: CC1(C)OB(c2cccc3c2sc2c(-c4cccc5c4sc4c(-c6ccc7oc8ccccc8c(=O)c7c6)cccc45)cccc23)OC1(C)C] | 799 |
| Exem- plary embodi- ment 15 | B11 | 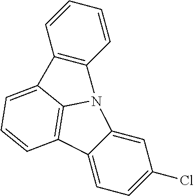 [SMILES: Clc1ccc2c3cccc4c5ccccc5n(c2c1)c43] | 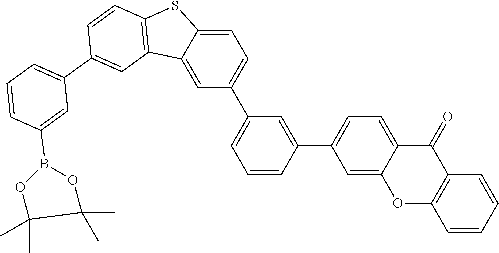 [SMILES: CC1(C)OB(c2cccc(-c3ccc4sc5ccc(-c6cccc(-c7ccc8c(=O)c9ccccc9oc8c7)c6)cc5c4c3)c2)OC1(C)C] | 769 |
| Exem- plary embodi- ment 16 | B12 | 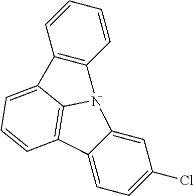 [SMILES: Clc1ccc2c3cccc4c5ccccc5n(c2c1)c43] | 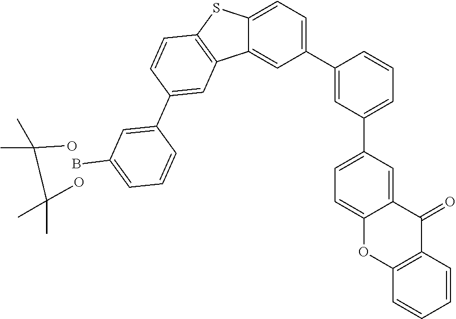 | 769 |
| Exem- plary embodi- ment 17 | B21 | 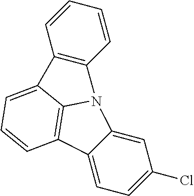 [SMILES: Clc1ccc2c3cccc4c5ccccc5n(c2c1)c43] | 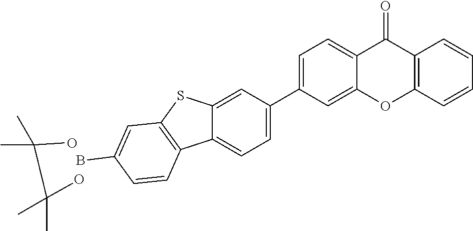 | 617 |
| Exem- plary embodi- ment 18 | B23 | 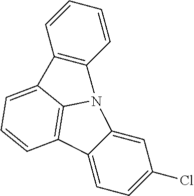 [SMILES: Clc1ccc2c3cccc4c5ccccc5n(c2c1)c43] | 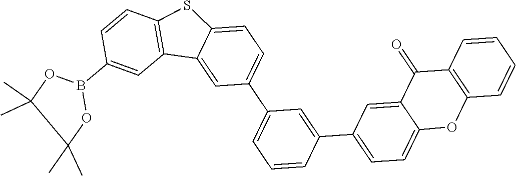 [SMILES: CC1(C)OB(c2ccc3sc4ccc(-c5cccc(-c6ccc7oc8ccccc8c(=O)c7c6)c5)cc4c3c2)OC1(C)C] | 693 |
| Exem- plary embodi- ment 19 | B24 | 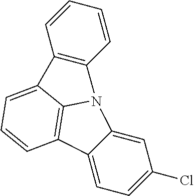 [SMILES: Clc1ccc2c3cccc4c5ccccc5n(c2c1)c43] | 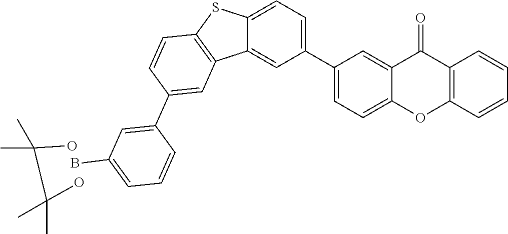 | 693 |
| Exem- plary embodi- ment 20 | B27 | 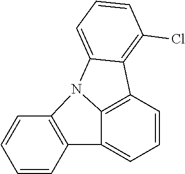 [SMILES: Clc1cccc2c1c1cccc3c4ccccc4n2c31] | 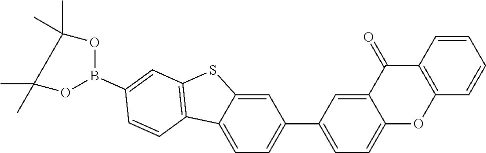 [SMILES: CC1(C)OB(c2ccc3c(c2)sc2cc(-c4ccc5oc6ccccc6c(=O)c5c4)ccc23)OC1(C)C] | 617 |

TABLE 6

| | Exem- | | | |
|---|---|---|---|---|
| | plary | | | |
| Exemplary | com- | | | |
| embodiment | pound | Raw material 1 | Raw material 2 | m/z |
| Exemplary embodiment 21 | B28 | 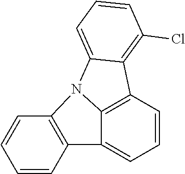 [SMILES: Clc1cccc2c1c1cccc3c4ccccc4n2c31] | 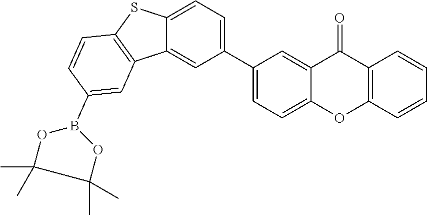 [SMILES: CC1(C)OB(c2ccc3sc4ccc(-c5ccc6oc7ccccc7c(=O)c6c5)cc4c3c2)OC1(C)C] | 617 |
| Exemplary embodiment 22 | B29 | 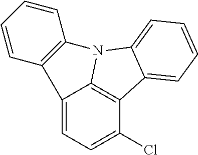 [SMILES: Clc1ccc2c3ccccc3n3c4ccccc4c1c23] | 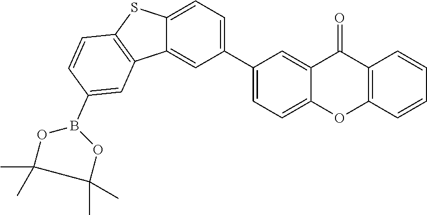 [SMILES: CC1(C)OB(c2ccc3sc4ccc(-c5ccc6oc7ccccc7c(=O)c6c5)cc4c3c2)OC1(C)C] | 617 |
| Exemplary embodiment 23 | B34 | 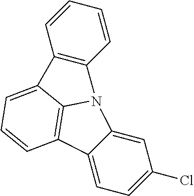 [SMILES: Clc1ccc2c3cccc4c5ccccc5n(c2c1)c43] | 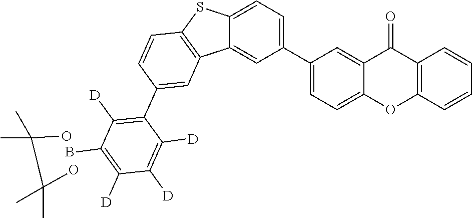 | 697 |
| Exemplary embodiment 24 | C1 | 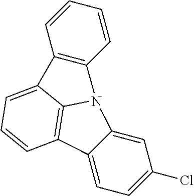 [SMILES: Clc1ccc2c3cccc4c5ccccc5n(c2c1)c43] | 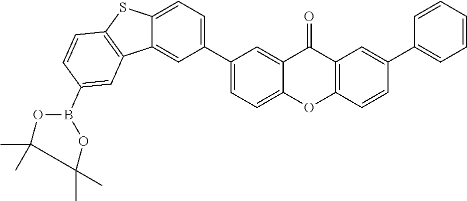 [SMILES: CC1(C)OB(c2ccc3sc4ccc(-c5ccc6oc7ccc(-c8ccccc8)cc7c(=O)c6c5)cc4c3c2)OC1(C)C] | 693 |
| Exemplary embodiment 25 | C2 | 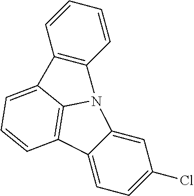 [SMILES: Clc1ccc2c3cccc4c5ccccc5n(c2c1)c43] | 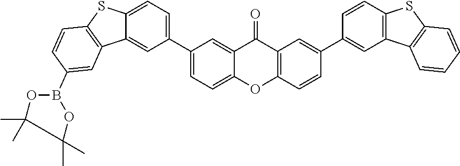 [SMILES: CC1(C)OB(c2ccc3sc4ccc(-c5ccc6oc7ccc(-c8ccc9sc%10ccccc%10c9c8)cc7c(=O)c6c5)cc4c3c2)OC1(C)C] | 799 |
| Exemplary embodiment 26 | C7 | 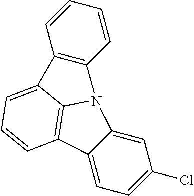 [SMILES: Clc1ccc2c3cccc4c5ccccc5n(c2c1)c43] | 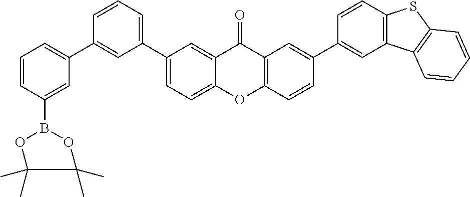 [SMILES: CC1(C)OB(c2cccc(-c3cccc(-c4ccc5oc6ccc(-c7ccc8sc9ccccc9c8c7)cc6c(=O)c5c4)c3)c2)OC1(C)C] | 769 |
| Exemplary embodiment 27 | C9 | 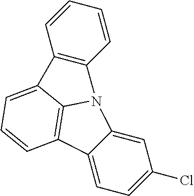 [SMILES: Clc1ccc2c3cccc4c5ccccc5n(c2c1)c43] | 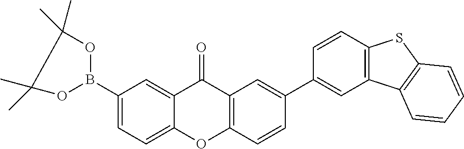 [SMILES: CC1(C)OB(c2ccc3oc4ccc(-c5ccc6sc7ccccc7c6c5)cc4c(=O)c3c2)OC1(C)C] | 617 |
| Exemplary embodiment 28 | C10 | 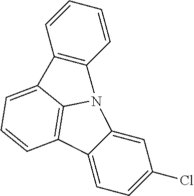 [SMILES: Clc1ccc2c3cccc4c5ccccc5n(c2c1)c43] | 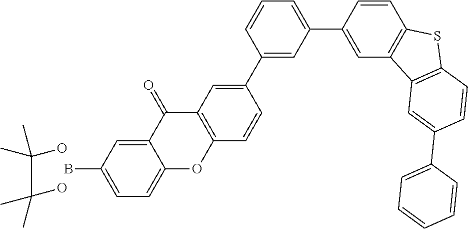 | 769 |
| Exemplary embodiment 29 | D7 | 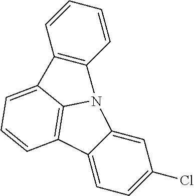 [SMILES: Clc1ccc2c3cccc4c5ccccc5n(c2c1)c43] | 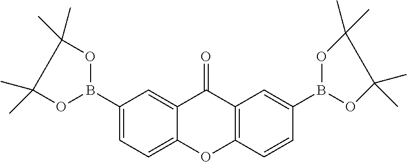 | 674 |
| Exemplary embodiment 30 | D8 | 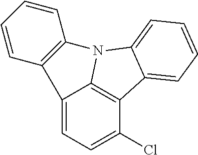 [SMILES: Clc1ccc2c3ccccc3n3c4ccccc4c1c23] | 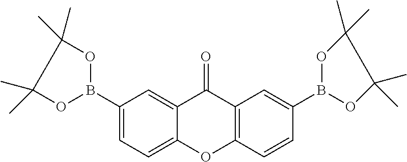 | 674 |

### Exemplary Embodiment 31

An organic light-emitting element of a bottom emission type was produced. The organic light-emitting element included a positive electrode, a hole injection layer, a hole transport layer, an electron-blocking layer, a light-emitting layer, a hole-blocking layer, an electron transport layer, an electron injection layer, and a negative electrode sequentially formed on a substrate.

First, an ITO film was formed on a glass substrate and was subjected to desired patterning to form an ITO electrode (positive electrode). The ITO electrode had a thickness of 100 nm. The substrate on which the ITO electrode was formed was used as an ITO substrate in the following process. Vacuum evaporation was then performed by resistance heating in a vacuum chamber at 1.33×10⁻⁴ Pa to continuously form an organic compound layer and an electrode layer shown in Table 11 on the ITO substrate. The counter electrode (a metal electrode layer, a negative electrode) had an electrode area of 3 mm².

TABLE 7

| | | | | Thickness |
|---|---|---|---|---|
| | Material | | | (nm) |
| Negative electrode | Al | | | 100 |
| Electron injection layer (EIL) | LiF | | | 1 |
| Electron transport layer (ETL) | ET2 | | | 20 |
| Hole-blocking layer (HBL) | ET11 | | | 20 |
| Light-emitting layer (EML) | Host | A1 | Mass ratio | 20 |
| | Guest | GD11 | B7:GD11 = | |
| | | | 90:10 | |

| | | |
|---|---|---|
| Electron-blocking layer (EBL) | HT19 | 15 |
| Hole transport layer (HTL) | HT3 | 30 |
| Hole injection layer (HIL) | HT16 | 5 |

The characteristics of the element were measured and evaluated. The light-emitting element had a maximum external quantum efficiency (E.Q.E.) of 13%. The present exemplary embodiment had a luminance decay rate ratio of 1.3 on the assumption that the time when the luminance decay rate of Comparative Example 1 described later reached 5% was 1.0.

A continuous operation test was performed at a current density of 100 mA/cm² to measure the time when the luminance decay rate reached 5%. In the present exemplary embodiment, with respect to measuring apparatuses, more specifically, the current-voltage characteristics were measured with a microammeter 4140B manufactured by Hewlett-Packard Co., and the luminous brightness was measured with BM7 manufactured by Topcon Corporation.

### Exemplary Embodiments 32 to 54 and Comparative Examples 1 and 2

Organic light-emitting elements were produced in the same manner as in Exemplary Embodiment 31 except that the compounds shown in Table 6 were used. Characteristics of the elements were measured and evaluated in the same manner as in Exemplary Embodiment 31. Table 6 shows the measurement results. With respect to the luminance decay rate ratio, in the same manner as in Exemplary Embodiment 1, on the assumption that the time when the luminance decay rate of the element of Comparative Example 1 reached 5% was 1.0, the time when the luminance decay rate of each element of the exemplary embodiments and the comparative examples reached 5% was shown as a relative ratio. Comparative Compounds 1-a and 1-b used as hosts in Comparative Examples 1 and 2 are the indolocarbazole derivatives 1-a and 1-b described in Patent Literature 1 and Patent Literature 2, respectively.

TABLE 8

| | | | | | | | | | Luminance |
|---|---|---|---|---|---|---|---|---|---|
| | | | | EML | | | | E.Q.E | decay rate |
| | HIL | HTL | ERL | Host | Guest | HBL | ETL | [%] | ratio |
| Exemplary embodiment 31 | HT16 | HT3 | HT19 | A1 | GD11 | ET11 | Et2 | 13 | 1.3 |
| Exemplary embodiment 32 | HT16 | HT3 | HT19 | A5 | GD11 | ET11 | ET2 | 14 | 1.3 |
| Exemplary embodiment 33 | HT16 | HT3 | HT19 | A11 | GD11 | ET11 | ET7 | 12 | 1.4 |
| Exemplary embodiment 34 | HT16 | HT3 | HT19 | A12 | GD11 | ET11 | ET7 | 12 | 1.4 |
| Exemplary embodiment 35 | HT16 | HT2 | HT19 | A13 | GD11 | ET11 | ET2 | 13 | 1.3 |
| Exemplary embodiment 36 | HT16 | HT3 | HT19 | A15 | GD11 | ET10 | ET2 | 14 | 1.3 |
| Exemplary embodiment 37 | HT16 | HT15 | HT19 | A21 | GD11 | ET10 | ET2 | 13 | 1.3 |
| Exemplary embodiment 38 | HT16 | HT3 | HT19 | A23 | GD11 | ET10 | ET2 | 14 | 1.3 |
| Exemplary embodiment 39 | HT16 | HT15 | HT19 | A27 | GD11 | ET11 | ET2 | 14 | 1.3 |
| Exemplary embodiment 40 | HT16 | HT2 | HT19 | A31 | GD11 | ET12 | ET7 | 13 | 1.3 |
| Exemplary embodiment 41 | HT16 | HT3 | HT19 | B4 | GD11 | ET11 | ET7 | 14 | 1.5 |
| Exemplary embodiment 42 | HT16 | HT3 | HT19 | B8 | GD11 | ET12 | ET2 | 14 | 1.6 |
| Exemplary embodiment 43 | HT16 | HT15 | HT19 | B11 | GD11 | ET12 | ET7 | 14 | 1.5 |
| Exemplary embodiment 44 | HT16 | HT15 | HT19 | B12 | GD11 | ET12 | ET7 | 13 | 1.4 |
| Exemplary embodiment 45 | HT16 | HT3 | HT19 | B21 | GD11 | ET11 | ET2 | 14 | 1.5 |
| Exemplary embodiment 46 | HT16 | HT3 | HT19 | B23 | GD11 | ET11 | ET2 | 14 | 1.4 |
| Exemplary embodiment 47 | HT16 | HT2 | HT19 | B24 | GD11 | ET11 | ET7 | 14 | 1.4 |
| Exemplary embodiment 48 | HT16 | HT15 | HT19 | B27 | GD11 | ET12 | ET2 | 13 | 1.5 |
| Exemplary embodiment 49 | HT16 | HT3 | HT19 | B28 | GD11 | ET10 | ET2 | 12 | 1.5 |
| Exemplary embodiment 50 | HT16 | HT3 | HT19 | B34 | GD11 | ET10 | ET2 | 14 | 1.4 |
| Exemplary embodiment 51 | HT16 | HT3 | HT19 | C1 | GD11 | ET11 | ET7 | 13 | 1.2 |
| Exemplary embodiment 52 | HT16 | HT15 | HT19 | C9 | GD11 | ET10 | ET7 | 13 | 1.2 |
| Exemplary embodiment 53 | HT16 | HT15 | HT19 | D7 | GD11 | ET10 | ET2 | 12 | 1.2 |
| Exemplary embodiment 54 | HT16 | HT3 | HT19 | D8 | GD11 | ET11 | ET7 | 12 | 1.3 |
| Comparative example 1 | HT16 | HT3 | HT19 | Comparative | GD11 | ET11 | ET2 | 10 | 1.0 |
| | | | | compound | | | | | |
| | | | | 1-a | | | | | |
| Comparative example 2 | HT16 | HT3 | HT19 | Comparative | GD11 | ET11 | ET2 | 12 | 0.8 |
| | | | | compound | | | | | |
| | | | | 1-b | | | | | |

As described above, as a host of a light-emitting layer, the use of an organic compound according to the present disclosure having an indolocarbazole ring and a xanthone ring bonded through a benzene ring, a dibenzothiophene ring, or a xanthone ring improves the compatibility between a host molecule and a guest molecule, reduces the intermolecular distance, and increases the energy transfer efficiency. It was found that this can provide an element with enhanced redox stability, high efficiency, and high durability.

An organic compound according to the present disclosure can be used for a phosphorescent layer to provide an efficient long-life light-emitting element.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2023-197266 filed Nov. 21, 2023, which is hereby incorporated by reference herein in its entirety.

What is claimed is:

1. An organic compound represented by formula [1]: 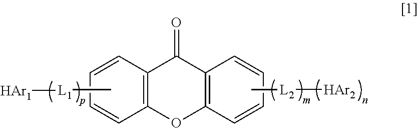
wherein p, m, and n are each independently 0 or 1, and p+m+n≥1,
L₁ is a divalent substituent derived from a substituted or unsubstituted biphenyl, terphenyl, dibenzothiophene, or combination of benzenes, dibenzothiophenes, and xanthones, wherein in a case of a number of the benzenes is q, a number of dibenzothiophenes is r, and a number of xanthones is s, each of the q, the r and the s independently is an integer of 0 or 1 or more, and q+r+s≥2,
L₂ is a monovalent or divalent substituent derived from a substituted or unsubstituted benzene, biphenyl, terphenyl, dibenzothiophene, or combination of benzenes, dibenzothiophenes, and xanthones, wherein in a case of a number of the benzenes is q, a number of dibenzothiophenes is r, and a number of xanthones is s, each of the q, the r and the s independently is an integer of 0 or 1 or more, and q+r+s≥2, and
HAr₁ and HAr₂ are each independently represented by any one of formulae [2-1] to [2-3], 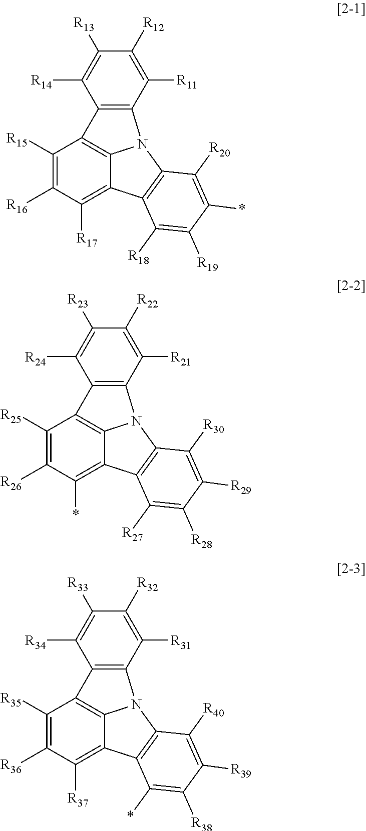
wherein R₁₀ to R₄₀ are selected from a hydrogen atom, a deuterium atom, a halogen atom, a substituted or unsubstituted alkyl group, a substituted or unsubstituted alkoxy group, and a substituted or unsubstituted silyl group.

2. The organic compound according to claim 1, wherein L₁ is a monovalent or divalent substituent derived from a combination of one or more substituted or unsubstituted dibenzothiophenes and two or more substituted or unsubstituted xanthones.

3. The organic compound according to claim 1, wherein L₁ is represented by any one of formulae [3-1] to [3-5]: 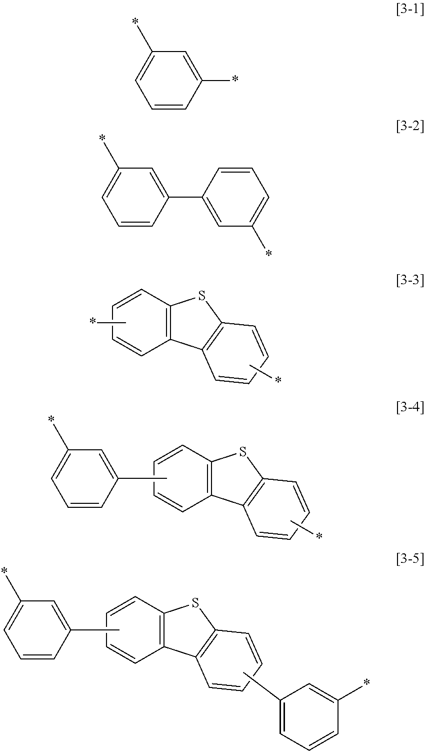
wherein * represents a binding position.

4. The organic compound according to claim 1, wherein n is 0.

5. An organic light-emitting element comprising:
a first electrode;
a second electrode; and
an organic compound layer between the first electrode and the second electrode,
wherein the organic compound layer contains the organic compound according to claim 1.

6. The organic light-emitting element according to claim 5, wherein
the organic compound layer includes a light-emitting layer, and
the light-emitting layer contains the organic compound.

7. The organic light-emitting element according to claim 6, wherein the light-emitting layer contains a phosphorescent compound.

8. The organic light-emitting element according to claim 7, wherein the light-emitting layer contains an electron transport compound.

9. The organic light-emitting element according to claim 6, wherein
the organic compound layer includes a second light-emitting layer disposed on the light-emitting layer serving as a first light-emitting layer, and
the second light-emitting layer emits light of a color different from a color of light emitted by the first light-emitting layer.

10. The organic light-emitting element according to claim 9, wherein the organic light-emitting element emits white light.

11. A display apparatus comprising a plurality of pixels, wherein at least one of the plurality of pixels includes the organic light-emitting element according to claim 5 and a transistor coupled to the organic light-emitting element.

12. A photoelectric conversion apparatus comprising:
an optical unit including a plurality of lenses;
an imaging element configured to receive light passing through the optical unit; and
a display unit configured to display an image taken by the imaging element,
wherein the display unit includes the organic light-emitting element according to claim 5.

13. Electronic equipment comprising:
a display unit including the organic light-emitting element according to claim 5;
a housing configured to be provided with the display unit; and
a communication unit provided in the housing and configured to communicate with an outside.

14. A lighting apparatus comprising:
a light source including the organic light-emitting element according to claim 5; and
a light-diffusing unit or an optical filter configured to transmit light emitted by the light source.

15. A moving body comprising:
a lamp including the organic light-emitting element according to claim 5; and
a body configured to be provided with the lamp.

16. An image-forming apparatus comprising:
a photosensitive member; and
an exposure light source configured to expose the photosensitive member,
wherein the exposure light source includes the organic light-emitting element according to claim 5.
